(19) United States
(12) Patent Application Publication
(10) Pub. No.: US 2017/0334947 A1
(43) Pub. Date: Nov. 23, 2017

(54) AFFINITY SEPARATION MATRIX FOR FAB REGION-CONTAINING PEPTIDE

(71) Applicant: KANEKA CORPORATION, Osaka-shi, Osaka (JP)

(72) Inventors: Dai MURATA, Takasago-shi (JP); Shinichi YOSHIDA, Takasago-shi (JP)

(73) Assignee: KANEKA CORPORATION, Osaka-shi, Osaka (JP)

(21) Appl. No.: 15/506,657

(22) PCT Filed: Aug. 27, 2015
(86) PCT No.: PCT/JP2015/074176

§ 371 Date: Feb. 24, 2017

(30) Foreign Application Priority Data
Aug. 28, 2014 (JP) 2014-174075

(51) Int. Cl. C07K 1/22; B01D 15/38; C07K 17/06; C07K 16/00; C07K 14/315

(52) U.S. Cl. CPC C07K 1/22; C07K 16/00; C07K 14/315; C07K 17/06; B01D 15/3809; C07B 2200/11; C07K 2317/55

(57) ABSTRACT

The objective of the present invention is to provide an affinity separation matrix having excellent adsorption performance and binding capacity to a peptide containing a Fab region of IgG, and a method for producing a Fab region-containing peptide using the affinity separation matrix. The affinity separation matrix according to the present invention is characterized in that a Fab region-binding peptide is immobilized as a ligand on a water-insoluble carrier in a density of 1.0 mg/mL-gel or more.

### TECHNICAL FIELD

The present invention relates to an affinity separation matrix having excellent adsorption performance and binding capacity to a peptide containing a Fab region of an immunoglobulin G, and a method for producing a Fab region-containing peptide using the affinity separation matrix.

### BACKGROUND ART

As one of important functions of a protein, a capability to specifically bind to a specific molecule is exemplified. The capability plays an important role in an immunoreaction and signal transduction in a living body. A technology utilizing the capability for purifying a useful substance has been actively developed. As one example of a protein which is actually utilized industrially, for example, Protein A affinity separation matrix has been used for capturing an antibody drug to be purified at one time from a culture of an animal cell with high purity (Non-patent Documents 1 and 2). Hereinafter, Protein A is abbreviated as “SpA” in some cases.

An antibody drug which has been mainly developed is a monoclonal antibody, and has been produced on a large scale by using recombinant cell cultivation technology. A “monoclonal antibody” means an antibody obtained from a clone derived from a single antibody-producing cell. Most of antibody drugs launched presently are classified into an immunoglobulin G in terms of a molecular structure. Hereinafter, an immunoglobulin G is abbreviated as “IgG” in some cases. In addition, an antibody drug consisting of an antibody fragment has been actively subjected to clinical development. An antibody fragment is an antibody derivative having a molecular structure obtained by fragmenting IgG. A plurality of antibody drugs consisting of a Fab fragment of IgG has been launched (Non-patent Document 3).

In an initial purification step in an antibody drug production, the above-described SpA affinity separation matrix is utilized. However, SpA is basically a protein which specifically binds to a Fc region of IgG. Thus, SpA affinity separation matrix cannot capture an antibody fragment which does not contain a Fc region. Accordingly, an affinity separation matrix capable of capturing an antibody fragment which does not contain a Fc region of IgG is highly required industrially.

A plurality of proteins which can bind to a region except for a Fc region of IgG have been already known (Non-patent Document 4). However, it is not true that an affinity separation matrix having such a protein as a ligand is generally industrially utilized in purification of an antibody drug similarly to SpA affinity separation matrix.

While there is no fact to be industrially used, CaptoL (trademark) having protein L as a ligand, KappaSelect (trademark) having a camel antibody as a ligand, Lammbda FabSelect or the like are known as an affinity separation matrix having a protein which binds to a Fab region of IgG as a ligand. However, the affinity separation matrixes recognize only one light chain subclass. Specifically, CaptoL (trademark) and KappaSelect (trademark) recognize only a κchain of Fab, and Lammbda FabSelect recognizes only a λchain of Fab. Accordingly, an affinity separation matrix which has a different recognition mechanism and which has a protein with a high universality in binding to a Fab region of IgG as a ligand is also desirable.

In addition, a protein which is referred to as Protein G and which is found in Streptococcus sp. classified into Group G can bind to IgG. Hereinafter, Protein G is referred to as “SpG”. A SpG affinity separation matrix product on which SpG is immobilized as a ligand has been commercially available (product name: “Protein-G Sepharose 4 Fast Flow” manufactured by GE Healthcare, Patent Document 1). It is known that SpG strongly binds to a Fc region of IgG and weakly binds to a Fab region (Non-patent Documents 4 and 5). However, since SpG has a weak binding affinity to a Fab region, the performance of a SpG affinity separation matrix product to maintain an antibody fragment which does not contain a Fc region and which contains a Fab region only is considered to be insufficient. It was therefore promoted to improve the binding affinity of SpG to a Fab region by introducing a mutation into SpG (Patent Document 2).

### PRIOR ART DOCUMENT

### Patent Document

Patent Document 1: JP S63-503032 T
Patent Document 2: JP 2009-195184 A

### Non-Patent Document

Non-patent Document 1: Hober S. et al., J. Chromatogr. B, 2007, vol. 848, pp. 40-47
Non-patent Document 2: Shukla A. A. et al., Trends Biotechnol., 2010, vol. 28, pp. 253-261
Non-patent Document 3: Nelson A. N. et al., Nat. Biotechnol., 2009, vol. 27, pp. 331-337
Non-patent Document 4: Bouvet P. J., Int. J. Immunopharmac., 1994, vol. 16, pp. 419-424
Non-patent Document 5: Derrick J. P., Nature, 1992, vol. 359, pp. 752-754

### DISCLOSURE OF THE INVENTION

### Problems to be Solved by the Invention

As described above, SpA affinity separation matrix on which Protein A (SpA) is immobilized as an affinity ligand for adsorbing an antibody to be purified has been conventionally put to practical use to adsorb an antibody to be purified, but such SpA affinity separation matrix has a specific adsorption performance to only a Fc region of IgG. However, an affinity separation matrix on which a ligand having an affinity for a Fab region of IgG is recently required, since a technology to use an antibody fragment as a drug has been developed. Protein G (SpG) is known as a protein having an affinity for not only a Fc region but also a Fab region, but an affinity for a Fab region thereof is lower than that for a Fc region. It is therefore examined to improve an affinity for a Fab region by introducing a mutation into SpG as the invention described in Patent Document 2. However, though an affinity of the SpG variant described in Patent Document 2 for a Fab region is improved in comparison with wild SpG, a carrier on which the SpG variant is immobilized does not demonstrate sufficient performance as an affinity separation matrix which is endurable to practical use due to low performance to adsorb an antibody fragment having a Fab region only.

Under the above-described circumstances, the objective of the present invention is to provide an affinity separation matrix having excellent adsorption performance and binding capacity to a peptide containing a Fab region of an immunoglobulin G, and a method for producing a Fab region-containing peptide using the affinity separation matrix.

### Means for Solving the Problems

The inventors of the present invention made extensive studies to solve the above problems. As a result, the inventors completed the present invention by finding that an affinity separation matrix useful for separating and purifying not only an immunoglobulin G but also an antibody fragment containing a Fab region can be obtained by immobilizing a Fab region-binding peptide having high affinity to a Fab region of an immunoglobulin G as a ligand on a water-insoluble carrier in a specific density.

Hereinafter, the present invention is described.

[1] An affinity separation matrix, wherein a Fab region-binding peptide is immobilized as a ligand on a water-insoluble carrier in a density of 1.0 mg/mL-gel or more.

[2] The affinity separation matrix according to the above [1], wherein the density is 5.0 mg/mL-gel or more.

[3] The affinity separation matrix according to the above [1] or [2], wherein an association constant of the Fab region-binding peptide to a Fab region is 10⁶ M⁻¹ or more.

[4] The affinity separation matrix according to any one of the above [1] to [3], wherein the Fab region-binding peptide is a variant of β1 domain of Protein G.

[5] The affinity separation matrix according to the above [4], wherein an amino acid sequence of the variant is an amino acid sequence derived from β1 domain of Protein G (SEQ ID NO: 3) with 3 or more substitutions of amino acid residues.

[6] The affinity separation matrix according to any one of the above [1] to [4], wherein the Fab region-binding peptide is selected from the following (1) to (3):

(1) a Fab region-binding peptide having an amino acid sequence corresponding to an amino acid sequence of SEQ ID NO: 3 derived from β1 domain of Protein G with substitution of one or more amino acid residues at positions selected from the 13^{th} position, the 15^{th} position, the 19^{th} position, the 30^{th} position and the 33^{rd} position, wherein a binding affinity to a Fab region of an immunoglobulin G is stronger than a binding affinity before introducing the substitution;

(2) a Fab region-binding peptide having the amino acid sequence specified in the (1) with deletion, substitution and/or addition of one or more amino acid residues in a region except for the 13^{th} position, the 15^{th} position, the 19^{th} position, the 30^{th} position and the 33^{rd} position, wherein a binding affinity to a Fab region of an immunoglobulin G is stronger than a binding affinity of a peptide having the amino acid sequence of SEQ ID NO: 3;

(3) a Fab region-binding peptide having an amino acid sequence with a sequence homology of 80% or more to the amino acid sequence specified in the (1), wherein a binding affinity to a Fab region of an immunoglobulin G is stronger than a binding affinity of a peptide having the amino acid sequence of SEQ ID NO: 3, provided that the amino acid residue substitution specified in the (1) at one or more positions selected from the 13^{th} position, the 15^{th} position, the 19^{th} position, the 30^{th} position and the 33^{rd} position is not further mutated in (3).

[7] The affinity separation matrix according to the above [6], wherein the amino acid residue at the 13^{th} position is substituted in the amino acid sequence specified in the (1).

[8] The affinity separation matrix according to the above [6], wherein the amino acid residue at the 13^{th} position is substituted by Thr or Ser in the amino acid sequence specified in the (1).

[9] The affinity separation matrix according to any one of the above [6] to [8], wherein the amino acid residue at the 30^{th} position is substituted by Val, Leu or Ile in the amino acid sequence specified in the (1).

[10] The affinity separation matrix according to any one of the above [6] to [9], wherein the amino acid residue at the 19^{th} position is substituted by Val, Leu or Ile in the amino acid sequence specified in the (1).

[11] The affinity separation matrix according to any one of the above [6] to [10], wherein the amino acid residue at the 33^{rd} position is substituted by Phe in the amino acid sequence specified in the (1).

[12] The affinity separation matrix according to any one of the above [6] to [11], wherein the amino acid residue at the 15^{th} position is substituted by Trp or Tyr in the amino acid sequence specified in the (1).

[13] The affinity separation matrix according to any one of the above [6] to [12], wherein a position of the deletion, substitution and/or addition of the amino acid residue is one or more positions selected from the 2^{nd} position, the 10^{th} position, the 18^{th} position, the 21^{st} position, the 22^{nd} position, the 23^{rd} position, the 24^{th} position, the 25^{th} position, the 27^{th} position, the 28^{th} position, the 31^{st}position, the 32^{nd} position, the 35^{th} position, the 36^{th} position, the 39^{th} position, the 40^{th} position, the 42^{nd} position, the 45^{th} position, the 47^{th} position and the 48^{th} position in the amino acid sequence specified in the (2).

[14] The affinity separation matrix according to any one of the above [6] to [13], wherein a position of the deletion, substitution and/or addition of the amino acid residue is N-terminal and/or C-terminal in the amino acid sequence specified in the (2).

[15] The affinity separation matrix according to any one of the above [6] to [14], wherein the sequence homology is 95% or more in the amino acid sequence specified in the (3).

[16] The affinity separation matrix according to any one of the above [1] to [15], wherein two or more domains formed by binding two or more of the Fab region-binding peptides are immobilized as a ligand.

[17] A method for producing a protein comprising a Fab region, comprising the steps of:

contacting the affinity separation matrix according to any one of the above [1] to [16] with a liquid sample comprising the protein comprising the Fab region; and

separating the protein comprising the Fab region bound on the affinity separation matrix from the affinity separation matrix.

### Effect of the Invention

The affinity separation matrix for a Fab region-containing peptide according to the present invention demonstrates high adsorption performance to not only a general antibody but also an antibody fragment which contains a Fab region but which does not contain a Fc region, since the matrix has high adsorption performance and binding capacity to a Fab region of IgG. Accordingly, an antibody fragment drug can be efficiently purified by the present invention matrix. Since the present invention can contribute to the practical realization of an antibody fragment drug under the situation that an antibody fragment drug has been actively developed recently due to the low production cost or the like, the present invention is industrially very useful.

### BRIEF DESCRIPTION OF THE DRAWINGS

FIG. 1 represents a method for preparing an expression plasmid of a wild SpG-β1.

FIG. 2 are charts of the binding reaction curves of wild SpG-β1 and a monomer of a SpG-β1 variant obtained by the present invention to a Fab region of anti-TNFα monoclonal antibody.

FIG. 3 are charts of the binding reaction curves of wild SpG-β1 and a dimer of a SpG-β1 variant obtained by the present invention to a Fab region of anti-TNFα monoclonal antibody.

FIG. 4 is a chart of chromatography obtained by purifying Fab from a mixture solution containing Fab using a column packed with the affinity separation matrix according to the present invention.

FIG. 5 is a photograph of the result of analyzing fractions of control and obtained by the above-described chromatography with acrylamide gel electrophoresis.

### MODE FOR CARRYING OUT THE INVENTION

The affinity separation matrix of the present invention is characterized in that a Fab region-binding peptide having a binding capability to a peptide having a Fab region of an immunoglobulin G (IgG) is immobilized as a ligand on a water-insoluble carrier in a specified density. The affinity separation matrix has the Fab region-binding peptide having a high binding affinity to a Fab region as a ligand, and furthermore a density of the ligand is adjusted to be high, thereby increasing a binding affinity to a Fab region as a matrix, and achieving a high adsorption performance to a Fab region-containing peptide and a high binding capacity per ligand density. The affinity separation matrix of the present invention has high adsorption performance and binding capacity to a Fab region-containing peptide, and thus is useful for purifying a Fab region-containing peptide.

In the present invention, the term “Fab region-binding peptide” refers to a peptide having a high binding capability to a Fab region of IgG. Specifically, a binding affinity to a Fab region of IgG is preferably 10⁶ M⁻¹ or more, and more preferably 10⁷ M⁻¹ or more, as an association constant (K_{A}). The binding force (affinity) of the Fab region-binding peptide according to the present invention to a Fab region of IgG can be evaluated, for example, by a biosensor such as Biacore system (GE Healthcare Bioscience) using a surface plasmon resonance principle and Octet system (Pall Corporation) using bio-layer interferometry, but is not restricted thereto.

With respect to a condition for measuring a binding capability to a Fab region, a binding signal at the time of binding to a Fab region of IgG may be detected. The binding capability can be easily measured at a constant temperature of 20 to 40° C. and in a neutral condition of pH 6 to 8.

For example, as a binding parameter, an association constant (K_{A}) and a dissociation constant (K_{D}) can be used (Nagata et al., “Real-Time Analysis Experimental Method for Interaction Between Biological Substances”, Springer-Verlag Tokyo, 1998, p. 41). For example, an association constant between the Fab region-binding peptide according to the present invention and a Fab fragment can be measured by immobilizing a Fab fragment on a sensor tip and adding the present invention peptide into a flow cell under a condition of a temperature of 25° C. and pH 7.4 in Biacore system. As the Fab region-binding peptide according to the present invention, a peptide of which association constant (K_{A}) is improved 2 or more times in comparison with wild Protein G is preferably used. The improvement rate of the preferably used Fab region-binding peptide is more preferably 5 times or more, even more preferably 10 times or more, even more preferably 20 times or more, and even more preferably 50 times or more and 10000 times or less.

In an experiment using Biacore system, an order of parameter may be largely changed depending on an experimental condition, analysis method and/or the kind of used IgG as a standard. As one of determination criteria in such a case, it is a standard whether an association constant for a Fab region is larger or not, when evaluating wild Protein G and a peptide having the amino acid sequence of SEQ ID NO: 3 under the same experimental conditions and analysis method. Wild Protein G is easily available as a commercial research reagent (for example, manufactured by Life Technologies). An association constant K_{A} for a Fab fragment of wild Protein G is about 10⁵ M⁻¹.

A IgG molecule as a binding partner is not particularly restricted as long as a binding to a Fab region thereof can be detected. However, it is preferred to use a fragmented and purified Fab region without a Fc region, since a binding to a Fc region is also detected in the case of using an immunoglobulin G containing a Fc region. The difference of affinity can be easily evaluated by obtaining bonding response curves to the same IgG molecule in the same measurement condition, analyzing the curves to obtain binding parameters, and comparing the parameters between a peptides before introducing the mutation and after introducing the mutation.

In the present invention, the term “peptide” means any molecules having a polypeptide structure. In the range of the “peptide”, not only a so-called protein but also a fragmented peptide and a peptide to which other peptide is bound through a peptide bond are included.

The term “immunoglobulin” is a glycoprotein produced by a B cell of a lymphocyte and has a function to recognize a molecule such as a specific protein to be bound. An immunoglobulin has not only a function to specifically bind to a specific molecule, i.e. antigen, but also a function to detoxify and remove an antigen-containing factor in cooperation with other biological molecule or cell. An immunoglobulin is generally referred to as “antibody”, and the name is inspired by such functions. All of immunoglobulins basically have the same molecular structure. The basic structure of an immunoglobulin is a Y-shaped four-chain structure consisting of two light chains and two heavy chains of polypeptide chains. A light chain (L chain) is classified into two types of λchain and κchain, and all of immunoglobulins have either of the types. A heavy chain (H chain) is classified into five types of γchain, μ chain, α chain, δ chain and δ chain, and an immunoglobulin is classified into isotypes depending on the kind of a heavy chain. An immunoglobulin G (IgG) is a monomer immunoglobulin, is composed of two heavy chains (γchains) and two light chains, and has two antigen-binding sites.

A lower half vertical part in the “Y” shape of an immunoglobulin is referred to as a “Fc region”, and an upper half “V” shaped part is referred to as a “Fab region”. A Fc region has an effector function to initiate a reaction after binding of an antibody to an antigen, and a Fab region has a function to bind to an antigen. A Fab region and Fc region of a heavy chain are bound to each other through a hinge part. Papain, which is a proteolytic enzyme and which is contained in papaya, decomposes a hinge part to cut into two Fab regions and one Fc region. The part close to the end of the “Y” shape in a Fab region is referred to as a “variable region (V region)”, since there are various changes in the amino acid sequence in order to bind to various antigens. A variable region of a light chain is referred to as a “VL region”, and a variable region of a heavy chain is referred to as a “VH region”. A Fc region and the other part in a Fab region except for a V region are referred to as a “constant region (C region)”, since there is relatively less change. A constant region of a light chain is referred to as a “CL region”, and a constant region of a heavy chain is referred to as a “CH region”. A CH region is further classified into three regions of CH1 to CH3. A Fab region of a heavy chain is composed of a VH region and CH1, and a Fc region of a heavy chain is composed of CH2 and CH3. There is a hinge part between CH1 and CH2. More specifically, SpG-β binds to a CH1 region (CH1γ) and a CL region of IgG, and particularly to a CH1 region mainly (Derrick J. P., Nature, 1992, vol. 359, pp. 752-754).

The Fab region-binding peptide used as a ligand of the affinity separation matrix according to the present invention binds to a Fab region of IgG. A Fab region-containing peptide to which the affinity separation matrix of the present invention binds may be a IgG molecule containing both of a Fab region and a Fc region or a derivative of a IgG molecule containing at least a Fab region as long as the peptide contains a Fab region. Such a IgG molecule derivative to be bound by the affinity separation matrix of the present invention is not particularly restricted as long as the derivative contains a Fab region. For example, the derivative is exemplified by a Fab fragment which is fragmented to only a Fab region of IgG, a chimera IgG prepared by replacing a part of domains of human IgG with a domain of IgG derived from other organism to be fused, IgG of which a sugar chain in a Fc region is subjected to molecular alteration, and a Fab fragment to which a drug covalently binds.

Protein G (SpG) is a protein derived from a cell wall of Streptococcus sp. classified into Group G. SpG has a capability to bind to an immunoglobulin G (IgG) of most mammals, strongly binds to a Fc region of IgG, and also weakly binds to a Fab region of IgG.

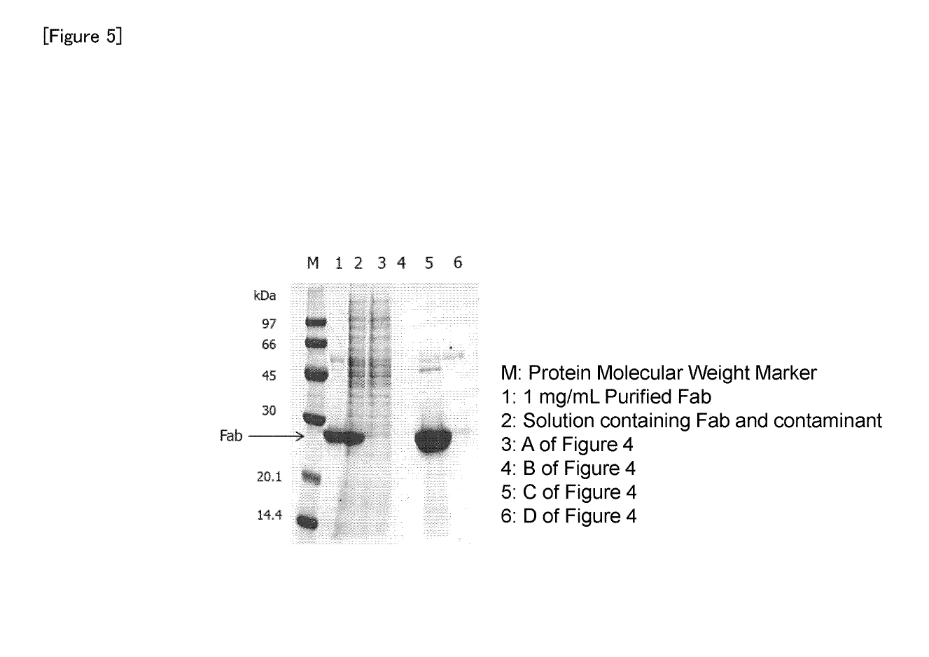

A SpG functional domain having a IgG-binding capability is referred to as “β domain, i.e. SpG-β”. The domain is referred to as β (B) domain or C domain (refer to Akerstrom et al., J. Biol. Chem., 1987, 28, p. 13388-, FIG. 5), but is referred to as “β domain” in the present invention in accordance with the definition of Fahnestock et al (Fahnestock et al., J. Bacteriol., 1986, 167, p. 870-). The details of the amino acid sequence of SpG-β are different depending on the kind and strain of a bacterium from which the SpG-β is derived. As the typical amino acid sequences, with respect to two β domains, β1 and β2, derived from Group G Streptococcus sp. GX7809 strain, the amino acid sequence of β1 domain (SpG-β1) is shown as SEQ ID NO: 1, and the amino acid sequence of β2 domain (SpG-β2) is shown as SEQ ID NO: 2. Each β domain of SpG is collectively referred to as Protein G-β domain (SpG-β), since the amino acid sequences of each β domain of SpG have high sequence identity with each other.

Alexander et al. found that the denaturation midpoint temperatures of SpG-β1 and SpG-β2 at pH 5.4 are respectively 87.5° C. and 79.4° C. (Alexander et al., Biochemistry, 1992, 31, p. 3597-). Accordingly, as one of preferred embodiments of the Fab region-binding peptide used as a ligand in the present invention, a variant of SpG-β1 (SEQ ID NO: 1) is exemplified as a comparison object in terms of thermal stability of a peptide, but a comparison object is not restricted thereto. In the present invention, the domain sequence of SEQ ID NO: 3, which corresponds to SpG-β1 (SEQ ID NO: 1) with substitution of the amino acid at the 1^{st} position, i.e. Asp, by Thr, is used as the SpG-β1 sequence for comparison. The 1^{st} position in SpG-β2 derived from the GX7809 strain is Thr, and the amino acid sequences on and after the 2^{nd} position of SEQ ID NO: 1 and SEQ ID NO: 2 are described as amino acid sequences of each domain of SpG in some documents. Accordingly, the above-described substitution in the SpG-β1 of SEQ ID NO: 1 does not have an effect on the affinity of the peptide for a Fab region. From the standpoint, in the Fab region-binding peptide (1), the amino acid sequence derived from SpG-β1 means the amino acid sequence of SpG-β1 or an amino acid sequence variant which is mutated in the range that an affinity of SpG-β1 for IgG-Fab is maintained.

The term “domain” means a unit of higher-order structure of a protein. A domain is composed of from dozens to hundreds of amino acid residues, and means a protein unit which can sufficiently serve some kind of a physicochemical or biochemical function.

The term “variant” of a protein or a peptide means a protein or peptide obtained by introducing at least one substitution, addition or deletion of an amino acid into a sequence of a wild protein or peptide.

As the Fab region-binding peptide having high binding affinity to a Fab region-containing peptide according to the present invention, a variant of a IgG-binding domain of Protein G, i.e. a SpG-β variant, is exemplified. The amino acid sequence before the mutation is preferably an amino acid sequence derived from SpG-β1 shown as SEQ ID NO: 3. However, a variant of a IgG-binding domain of other Protein G, such as SEQ ID NO: 2, also has a high sequence identity; therefore, a peptide obtained as a result of adding a mutation to an amino acid sequence of a wild IgG-binding domain, such as SpG-β2 variant, can be also used in the present invention.

Specifically, the Fab region-binding peptide according to the present invention is exemplified by the Fab region-binding peptides (1) to (3).

In the present invention, a mutation to substitute an amino acid is described by adding a wild or non-mutated amino acid residue before the number of substituted position and adding a mutated amino acid residue after the number of substituted position. For example, the mutation to substitute Gly at 29^{th} position by Ala is described as G29A.

The Fab region-binding peptide (1) used as a ligand of the affinity separation matrix according to the present invention is a peptide having an amino acid sequence which corresponds to an amino acid sequence derived from pi domain of Protein G (SEQ ID NO: 3) with substitution of one or more amino acids at positions selected from the 13^{th} position, the 15^{th} position, the 19^{th} position, the 30^{th} position and the 33^{rd} position, wherein a binding affinity to a Fab region of an immunoglobulin G is stronger than a binding affinity before introducing the amino acid substitution. In the present disclosure, the peptide before introducing the substitution means a pi domain of wild SpG having SEQ ID NO: 1 or a peptide having an amino acid sequence of SEQ ID NO: 3.

The essential substitution position of the amino acid residue of the Fab region-binding peptide (1) according to the present invention is any one or more positions of the 13^{th} position (Lys), the 15^{th} position (Glu), the 19^{th} position (Glu), the 30^{th} position (Phe) and the 33^{rd} position (Tyr) in the amino acid sequence of SEQ ID NO: 3. The number of the mutations is preferably 2 or more, and more preferably 3 or more.

The Fab region-binding peptide (1) according to the present invention has an amino acid sequence which corresponds to an amino acid sequence (SEQ ID NO: 3) of a β1 domain of Protein G with substitution of an amino acid residue at one or more positions selected from the 13^{th} position, the 15^{th} position, the 19^{th} position, the 30^{th} position and the 33^{rd} position. The kind of an amino acid for mutation, such as a substitution by a non-protein-constituting amino acid and a non-natural amino acid, is not particularly restricted, and a natural amino acid can be preferably used in terms of genetic engineering production. A natural amino acid is classified into the categories of a neutral amino acid; an acidic amino acid such as Asp and Glu; and a basic amino acid such as Lys, Arg and His. A neutral amino acid is classified into the categories of an aliphatic amino acid; an imino acid such as Pro; and an aromatic amino acid such as Phe, Tyr and Trp. An aliphatic amino acid is classified into the categories of Gly; Ala; a branched amino acid such as Val, Leu and Ile; a hydroxy amino acid such as Ser and Thr; a sulfur-containing amino acid such as Cys and Met; and an acid amide amino acid such as Asn and Gln. Since Tyr has a phenolic hydroxyl group, Tyr may be classified into not only an aromatic amino acid but also a hydroxy amino acid. From another viewpoint, a natural amino acid may also be classified into the categories of a nonpolar amino acid with high hydrophobicity, such as Gly, Ala, Val, Leu, Ile, Trp, Cys, Met, Pro and Phe; a neutral polar amino acid such as Asn, Gln, Ser, Thr and Tyr; an acidic polar amino acid such as Asp and Glu; and a basic polar amino acid such as Lys, Arg and His. When a binding affinity of a peptide in which the amino acid residue at the above-described position is substituted to a Fab region is improved, it is highly possible that a binding affinity of a peptide in which the substituted amino acid is further substituted by an amino acid classified into the same category to a Fab region is similarly improved.

The substitution mutation in the Fab region-binding peptide of the present invention preferably includes a mutation in which the amino acid residue at the 13^{th} position is substituted by Thr or Ser, and/or a mutation in which the amino acid residue at the 15^{th} position is substituted by Tyr or Trp, and/or a mutation in which the amino acid residue at the 19^{th} position is substituted by Val, Leu or Ile, and/or a mutation in which the amino acid residue at the 30^{th} position is substituted by Val, Leu or Ile, and/or a mutation in which the amino acid residue at the 33^{rd} position is substituted by Phe. The amino acid residue at the 13^{th} position is more preferably substituted by Thr, the amino acid residue at the 15^{th} position is more preferably substituted by Tyr, the amino acid residue at the 19^{th} position is more preferably substituted by Ile, and the amino acid residue at the 30^{th} position is more preferably substituted by Leu.

The Fab region-binding peptide (2) used as a ligand of the affinity separation matrix according to the present invention has the amino acid sequence specified in the above-described Fab region-binding peptide (1) with deletion, substitution and/or addition of one or more amino acid residues in a region except for the 13^{th} position, the 15^{th} position, the 19^{th} position, the 30^{th} position and the 33^{rd} position, wherein a binding affinity to a Fab region of an immunoglobulin G is stronger than a binding affinity of a peptide having the amino acid sequence of SEQ ID NO: 3.

The range of “one or more” in the phrase “amino acid sequence with deletion, substitution and/or addition of one or more amino acid residues” is not particularly restricted as long as the Fab region-binding peptide with deletion or the like has a strong binding affinity to a Fab region of IgG. The above-described range of “one or more” is exemplified by 1 or more and 30 or less, preferably 1 or more and 20 or less, more preferably 1 or more and 10 or less, even more preferably 1 or more and 7 or less, even more preferably 1 or more and 5 or less, and particularly preferably 1 or more and 3 or less, 1 or 2, or 1.

In the Fab region-binding peptide (2), as the position of deletion, substitution and/or addition in the amino acid sequence, one or more positions selected from the 2^{nd} position, the 10^{th} position, the 18^{th} position, the 21^{st} position, the 22^{nd} position, the 23^{rd} position, the 24^{th} position, the 25^{th} position, the 27^{th} position, the 28^{th} position, the 31^{st} position, the 32^{nd} position, the 35^{th} position, the 36^{th} position, the 39^{th} position, the 40^{th} position, the 42^{nd} position, the 45^{th} position, the 47^{th} position and the 48^{th} position are preferred, and one or more positions selected from the 10^{th} position, the 18^{th} position, the 21^{st} position, the 25^{th} position, the 28^{th} position, the 35^{th} position, the 39^{th} position and the 47^{th} position are more preferred. The kind of amino acid by which the amino acid residue at the above-described position is substituted is not particularly restricted, and the 2^{nd} position is preferably Arg, the 10^{th} position is preferably Arg, the 18^{th} position is preferably Ala, the 21^{st} position is preferably Ile, Ala or Asp, the 22^{nd} position is preferably Asn or Glu, the 23^{rd} position is preferably Thr or Asp, the 24^{th} position is preferably Thr, the 25^{th} position is preferably Ser or Met, the 27^{th} position is preferably Asp or Gly, the 28^{th} position is preferably Arg, Asn or Ile, the 31^{st} position is preferably Arg, the 32^{nd} position is preferably Arg, the 35^{th} position is preferably an aromatic amino acid such as Phe and Tyr, the 36^{th} position is preferably Gly, the 39^{th} position is preferably Leu or Ile, the 40^{th} position is preferably Val or Glu, the 42^{nd} position is preferably Leu, Val or Gln, the 45^{th} position is preferably Phe, the 47^{th} position is preferably His, Asn, Ala, Gly or Tyr, and the 48^{th} position is preferably Thr. In particular, the 2^{nd} position is preferably Arg, the 10^{th} position is preferably Arg, the 18^{th} position is preferably Ala, the 21^{st} position is preferably Ala or Asp, the 39^{th} position is preferably Leu or Ile, and the 47^{th} position is preferably Ala.

Also, the suitable position to be substituted is exemplified by one or more positions selected from the 6^{th} position, the 7^{th} position, the 24^{th} position, the 28^{th} position, the 29^{th} position, the 31^{st} position, the 35^{th} position, the 40^{th} position, the 42^{nd} position and the 47^{th} position, at which the kind of amino acid is different between wild SpG-β and a publicly-known SpG-β variant. From the viewpoint of binding activity and structure maintenance, the position of deletion and/or addition is preferably a N-terminal and/or a C-terminal.

The Fab region-binding peptide (3) used as a ligand of the affinity separation matrix according to the present invention has an amino acid sequence with a sequence homology of 80% or more with the amino acid sequence specified in the (1), wherein a binding affinity to a Fab region of an immunoglobulin G is stronger than a binding affinity of a peptide having the amino acid sequence of SEQ ID NO: 3, provided that the amino acid substitution specified in the (1) at one or more positions selected from the 13^{th} position, the 15^{th} position, the 19^{th} position, the 30^{th} position and the 33^{rd} position is not further mutated in (3).

The sequence identity is more preferably 85% or more, even more preferably 90% or more, and particularly preferably 95% or more. The sequence identity can be measured by a program for amino acid sequence multiple alignment, such as Clustal (http://www.clustal.org/omega/).

Even when the number of amino acids in the amino acid sequence of the Fab region-binding peptides (2) and (3) is different from the number before introduction of the mutation or the number of the amino acid sequence of the Fab region-binding peptide (1) as a result of further mutation, a skilled person can easily identify the position corresponding to the 13^{th} position, the 15^{th} position, the 19^{th} position, the 30^{th} position and the 33^{rd} position of SEQ ID NO: 3. Specifically, the position can be confirmed by aligning the sequences using a program for amino acid sequence multiple alignment: Clustal (http://www.clustal.org/omega/).

Protein G, i.e. SpG, is a protein which contains 2 or 3 IgG-binding domains in the form of tandem line. As one of the embodiments, the Fab region-binding peptide used as a ligand of the affinity separation matrix according to the present invention may be a multimer of 2 or more monomers or single domains of the Fab region-binding peptide connected each other. The number of the monomers or single domains is preferably 3 or more, more preferably 4 or more, and even more preferably 5 or more. With respect to the upper limit of the number of connected domains, the number may be 10 or less, preferably 8 or less, and more preferably 6 or less. Such a multimer may be a homomultimer in which one kind of Fab region-binding peptides are connected, such as homodimer and homotrimer, or a heteromultimer in which two or more kinds of Fab region-binding peptides are connected, such as heterodimer and heterotrimer. As described above, one or more amino acids may be added also to a multimer containing a plurality of domains. The position to be added is preferably a N-terminal and a C-terminal. As one of the embodiments, Cys may be added to a C-terminal of a two-domain type Fab region-binding peptide.

A method for connecting monomer proteins used as a ligand of the affinity separation matrix according to the present invention is exemplified by a connecting method through one or more amino acid residues, but is not restricted thereto. The number of the amino acid residue for connection is not particularly restricted, and is preferably 20 residues or less, and more preferably 15 residues or less. It is preferred to use a sequence which connects β1 and β2 or β2 and β3 of wild SpG. From another point of view, it is preferred that the amino acid residue for connection does not destabilize a three dimensional structure of a monomer protein.

As one of the embodiments, the ligand of the affinity separation matrix of the present invention may be a fusion peptide characterized in that the Fab region-binding peptide or a peptide multimer having two or more Fab region-binding peptides connected each other is fused with other peptide having a different function as one component. In other words, the amino acid sequence of the Fab region-binding peptide according to the present invention may contain any one of the amino acid sequences (1) to (3) and other amino acid sequence or other compound to be bound. A fusion peptide is exemplified by a peptide fused with albumin or GST, i.e. glutathione S-transferase, but is not restricted to the examples. In addition, peptides fused with a nucleic acid such as DNA aptamer, a drug such as an antibiotic or a polymer such as PEG, i.e. polyethylene glycol, are also included in the range of the present invention as long as such a fusion peptide is useful for the affinity separation matrix according to the present invention. However, it is preferred that the amino acid sequence of the Fab region-binding peptide according to the present invention is composed of any one of the amino acid sequences (1) to (3). Even in such a case, the Fab region-binding peptide according to the present invention may be immobilized on a water-insoluble carrier through a linker group as described later, and the Fab region-binding peptides may be connected each other through a linker group in the case of a multimer.

The Fab region-binding peptide used as a ligand of the affinity separation matrix according to the present invention may be obtained as a fusion peptide fused with a publicly-known protein which beneficially has an action to assist the expression of the protein or to facilitate the purification of the protein. In other words, it is possible to obtain a microorganism or cell containing at least one recombinant DNA encoding a fusion peptide containing the Fab region-binding peptide according to the present invention. The above-described protein is exemplified by a maltose-binding protein (MBP) and a glutathione S-transferase (GST), but is not restricted to the exemplified proteins.

Site-specific mutagenesis for modifying the DNA encoding the peptide of SEQ ID NO: 3 in order to obtain the Fab region-binding peptide used as a ligand of the affinity separation matrix according to the present invention can be carried out using recombinant DNA technology, PCR method or the like as follows.

For example, mutagenesis by recombinant DNA technology can be carried out as follows: in the case where there are suitable restriction enzyme recognition sequences on both sides of a target mutagenesis site in the gene encoding the Fab region-binding peptide, cassette mutagenesis method can be carried out in which method a region containing the target mutagenesis site is removed by cleaving the restriction enzyme recognition sites with the above-described restriction enzymes and then a mutated DNA fragment is inserted. Into the mutated DNA fragment, mutation is introduced only at the target site by a method such as chemical synthesis.

For example, site-directed mutagenesis by PCR can be carried out by double primer mutagenesis. In double primer mutagenesis, PCR is carried out by using a double-stranded plasmid encoding the Fab region-binding peptide as a template, and using two kinds of synthesized oligo primers which contain complementary mutations in the + strand and − strand.

A DNA encoding a multimer peptide can be produced by ligating the desired number of DNAs each encoding the monomer peptide (single domain) used as a ligand of the affinity separation matrix according to the present invention to one another in tandem. For example, with respect to connecting method for the DNA encoding the multimer peptide, a suitable restriction enzyme site is introduced in the DNA sequence and double-stranded DNA fragments cleaved with a restriction enzyme are ligated using a DNA ligase. One restriction enzyme site may be introduced or a plurality of restriction enzyme sites of different types may be introduced. When the base sequences encoding each monomer peptide in the DNA encoding the multimer peptide are the same, homologous recombination may be possibly induced in a host. Thus, the sequence identity between base sequences of DNAs encoding the monomer peptides to be connected may be 90% or less, preferably 85% or less, more preferably 80% or less, and even more preferably 75% or less. The identity of a base sequence can be also determined by an ordinary method similarly to an amino acid sequence.

The Fab region-binding peptide used as a ligand in the present invention can be produced by preparing an expression vector which contains a base sequence encoding the above-described peptide of the present invention or a part of the amino acid sequence of the peptide and a promoter which can be operably linked to the base sequence to function in a host, obtaining a transformant by introducing the prepared recombinant vector into a host cell, culturing the transformant in a medium to allow the transformant to produce and accumulate the peptide of the present invention in the cultured bacterial cell (including the periplasmic space of the bacterial cell) or in the culture liquid (outside the bacterial cell), and collecting the desired peptide from the culture. In general, the gene encoding the target peptide is linked or inserted to a suitable vector. The vector into which the gene is inserted is not particularly restricted as long as the vector is capable of autonomous replication in a host. As such a vector, a plasmid DNA or a phage DNA can be used. For example, in the case of using Escherichia coli as a host, a pQE series vector (manufactured by QIAGEN), a pET series vector (manufactured by Merck), a pGEX series vector (manufactured by GE Healthcare Bioscience) or the like can be used.

A method for introducing the recombinant DNA into a host is exemplified by a method using a calcium ion, electroporation method, spheroplast method, lithium acetate method, agrobacterium infection method, particle gun method and polyethylene-glycol method, but is not restricted thereto. A method for expressing the function of the obtained gene in a host is also exemplified by a method in which the gene according to the present invention is implanted into a genome (chromosome). A host cell is not particularly restricted, and bacteria (eubacteria) such as Escherichia coli, Bacillus subtilis, Brevibacillus, Staphylococcus, Streptococcus, Streptomyces and Corynebacterium can be preferably used in terms of mass production in a low cost.

In addition, the Fab region-binding peptide used as a ligand in the present invention can also be produced by culturing the above-described transformant in a medium to allow the transformant to produce and accumulate a fusion protein containing the peptide of the present invention in the cultured bacterial cell (including the periplasmic space of the cell) or in the culture liquid (outside the cell), collecting the fusion peptide from the culture, cleaving the fusion peptide with a suitable protease, and collecting the desired peptide.

The transformant of the present invention can be cultured in a medium in accordance with a common method for culturing a host cell. The medium used for culturing the obtained transformant is not particularly restricted as long as the medium enables high yield production of the present invention peptide with high efficiency. Specifically, carbon source and nitrogen source, such as glucose, sucrose, glycerol, polypeptone, meat extract, yeast extract and casamino acid, can be used. In addition, an inorganic salt such as potassium salt, sodium salt, phosphate, magnesium salt, manganese salt, zinc salt and iron salt is added as required. In the case of an auxotrophic host cell, a nutritional substance necessary for the growth thereof may be added. In addition, an antibiotic such as penicillin, erythromycin, chloramphenicol and neomycin may be added as required.

Furthermore, in order to inhibit the degradation of the target peptide caused by a host-derived protease present inside or outside the bacterial cell, a publicly-known protease inhibitor and/or other commercially available protease inhibitor may be added in an appropriate concentration. The publicly-known protease inhibitor is exemplified by phenylmethane sulfonyl fluoride (PMSF), benzamidine, 4-(2-aminoethyl)-benzenesulfonyl fluoride (AEBSF), antipain, chymostatin, leupeptin, Pepstatin A, phosphoramidon, aprotinin and ethylenediaminetetraacetic acid (EDTA).

In order to obtain rightly folded Fab region-binding peptide used as a ligand in the present invention, for example, a molecular chaperone such as GroEL/ES, Hsp70/DnaK, Hsp90 and Hsp104/ClpB may be used. For example, such a molecular chaperone is co-existed with the present invention peptide by coexpression or as a fusion protein. As a method for obtaining rightly folded present invention peptide, addition of an additive for assisting right folding into the medium and culturing at a low temperature are exemplified, but the method is not restricted thereto.

The medium for culturing transformant produced from an Escherichia coli as a host is exemplified by LB medium containing triptone 1%, yeast extract 0.5% and NaCl 1%, 2×YT medium containing triptone 1.6%, yeast extract 1.0% and NaCl 0.5%, or the like.

For example, the transformant may be aerobically cultured in an aeration-stirring condition at a temperature of 15 to 42° C., preferably 20 to 37° C., for from several hours to several days. As a result, the peptide of the present invention is accumulated in the cultured cell (including the periplasmic space of the cell) or in the culture liquid (outside the cell) to be recovered. In some cases, the culturing may be performed anaerobically without aeration. In the case where a recombinant peptide is secreted, the produced recombinant peptide can be recovered after the culture period by separating the supernatant containing the secreted peptide using a common separation method such as centrifugation and filtration from the cultured cell. In addition, in the case where the peptide is accumulated in the cultured cell (including the periplasmic space), the peptide accumulated in the cell can be recovered, for example, by collecting the bacterial cell from the culture liquid by centrifugation, filtration or the like, and then disrupting the bacterial cell by sonication method, French press method or the like, or solubilizing the bacterial cell by adding a surfactant or the like.

A method for purifying the peptide used as a ligand in the present invention can be carried out by any one or an appropriate combination of techniques such as affinity chromatography, cation or anion exchange chromatography, gel filtration chromatography and the like. It can be confirmed whether the obtained purified substance is the target peptide or not by an ordinary method such as SDS polyacrylamide gel electrophoresis, N-terminal amino acid sequence analysis and Western blot analysis.

The affinity separation matrix of the present invention can be prepared by immobilizing the Fab region-binding peptide having an excellent binding capability to a Fab region on a water-insoluble carrier. Such a water-insoluble carrier usable in the present invention is not particularly restricted, and is exemplified by an inorganic carrier such as glass beads and silica gel; synthetic polymer carrier such as cross-linked polyvinyl alcohol, cross-linked polyacrylate, cross-linked polyacrylamide and cross-linked polystyrene; a polysaccharide carrier such as crystalline cellulose, cross-linked cellulose, cross-linked agarose and cross-linked dextran; and a composite carrier obtained from the combination of the above carriers, such as an organic-organic composite carrier and an organic-inorganic composite carrier. The commercial product thereof is exemplified by porous cellulose gel GCL2000, Sephacryl (registered trademark) S-1000 prepared by crosslinking allyl dextran and methylene bisacrylamide through a covalent bond, an acrylate carrier Toyopearl (registered trademark), a cross-linked agarose carrier Sepharose (registered trademark) CL4B, and a cross-linked cellulose carrier Cellufine (registered trademark). However, it should be noted that the water-insoluble carrier usable in the present invention is not restricted to the carriers exemplified as the above.

It is preferred that the water-insoluble carrier usable in the present invention has large surface area and that the carrier is porous with a large number of fine pores having a suitable size in terms of a purpose and method for using the affinity separation matrix according to the present invention. The carrier may have any form such as beads, monolith, fiber and film (including hollow fiber), and any form can be selected.

With respect to a method for immobilizing the ligand, for example, the ligand can be bound to a carrier by a conventional coupling method utilizing an amino group, a carboxy group or a thiol group of the ligand. Such a coupling method is exemplified by an immobilization method including activation of a carrier by a reaction with cyanogen bromide, epichlorohydrin, diglycidyl ether, tosyl chloride, tresyl chloride, hydrazine, sodium periodate or the like, or introduction of a reactive functional group such as maleimide and NHS ester on the carrier surface, and the coupling reaction between the resulting carrier and a compound to be immobilized as a ligand; and an immobilization method by condensation and crosslinking which method includes adding a condensation reagent such as carbodiimide or a reagent having a plurality of functional groups in the molecule, such as glutaraldehyde, into a mixture containing a carrier and a compound to be immobilized as a ligand.

A spacer molecule composed of a plurality of atoms may be introduced between the ligand and carrier. Alternatively, the ligand may be directly immobilized on the carrier. Accordingly, the Fab region-binding peptide according to the present invention may be chemically modified for immobilization, or may have an additional peptide containing 1 or more and 100 or less amino acid residues useful for immobilization as a linker group. Such an amino acid useful for immobilization is exemplified by an amino acid having a functional group useful for a chemical reaction for immobilization in a side chain, and specifically exemplified by Lys having an amino group in a side chain and Cys having a thiol group in a side chain. The number of the amino acid residue contained in the above-described peptide linker group is preferably 50 or less, more preferably 40 or less or 20 or less, and even more preferably 10 or less. Since the binding capability of the peptide according to the present invention to a Fab region is principally maintained in a matrix prepared by immobilizing the peptide as a ligand in the present invention, any modification and change for immobilization are included in the range of the present invention.

The affinity separation matrix according to the present invention is characterized in that the Fab region-binding peptide is immobilized as a ligand on a water-insoluble carrier in a density of 1.0 mg/mL-gel or more, in other words, in a density of 1.0 mg or more per 1 mL of gelatinous matrix. As a result, the affinity separation matrix according to the present invention exhibits an excellent affinity for a Fab region-containing peptide and excellent adsorption performance and binding capacity to a Fab region-containing peptide.

An amount of the Fab region-binding peptide immobilized on the affinity separation matrix according to the present invention can be adjusted by an amount of a functional group to which the peptide is bound and which is introduced into the water-insoluble carrier, an amount of the Fab region-binding peptide to be reacted with the water-insoluble carrier, a reaction condition, or the like.

The ligand density corresponds to a value obtained by dividing an amount of the ligand immobilized on the affinity separation matrix by a volume of the gelatinous affinity separation matrix. The ligand density is preferably 1.5 mg/mL-gel or more, more preferably 2.0 mg/mL-gel or more, and even more preferably 5.0 mg/mL-gel or more. The upper limit of the ligand density is not particularly restricted; and when the ligand density is higher, the adsorption performance and binding capacity of the matrix to a Fab region-containing peptide are more excellent. However, since it may be possibly difficult to produce a matrix having excessively high ligand density, the ligand density is preferably 40 mg/mL-gel or less.

A volume of the affinity separation matrix used as a basis for calculating the ligand density means a volume of the matrix on which the ligand is immobilized in a gel state that the matrix is capable of coupling and retaining a Fab region-containing peptide. For example, the volume can be measured after suspending the affinity separation matrix according to the present invention in water, a neutral phosphate buffer solution or the like and transferring the suspension into a measuring tool such as a graduated cylinder, then sufficiently standing the measuring device still until an apparent volume is not decreased any further. It may take time to stand still depending on the material of the matrix. In such a case, it is also possible to lightly tap the measuring container until an apparent volume is not decreased any further, then stand the measuring container still to measure the volume. In a case of a commercial prepacked carrier, since a matrix with a predetermined volume is packed in a column, the volume is defined as a volume of the matrix.

A mass of the ligand immobilized on the affinity separation matrix can be calculated from the difference between a mass of the ligand which is reacted with a water-insoluble carrier and a mass of the ligand recovered without being immobilized after the immobilization reaction. The masses of the ligands may be directly weighed or may be indirectly measured by absorbance measurement or the like. For example, a mass of the ligand immobilized on the affinity separation matrix can be obtained by preliminarily calculating a ligand amount in a ligand solution to be reacted with a water-insoluble carrier by absorbance measurement, then calculating an unreacted ligand amount by absorbance measurement of an unreacted ligand solution after the immobilization reaction, and calculating the difference of the measured values. A mass of the ligand can be also evaluated by using an absorption coefficient calculated from the amino acid sequence. When the ligand can be directly weighed, a mass of the ligand can be also evaluated by using an absorption coefficient obtained by preparing the solution thereof.

Also, a mass of the ligand immobilized on the affinity separation matrix can be also obtained by using a quantitative determination of protein using a bicinchoninic acid (BCA) reagent. For example, the affinity separation matrix suspended in water is put into a measurement tool such as a graduated cylinder, and the measurement tool is sufficiently stood still until an apparent volume is not decreased any further, then a volume is measured. Further, a BCA reagent is mixed thereto, the mixture is reacted for a certain time, and then an absorbency at 562 nm is measured, whereby an amount of immobilized ligand per the volume of weighed affinity separation matrix can be evaluated. A mass of the ligand in the above case can be evaluated by previously measuring the ligand mass-dependent value of absorbency at 562 nm.

A method for evaluating the ligand density is exemplified as the above, but is not restricted thereto.

An adsorption performance to a Fab region-containing peptide can be demonstrated as an index of a ratio of an amount of the Fab region-containing peptide which can be recovered from the affinity separation matrix after the steps of loading of the Fab region-containing peptide on the affinity separation matrix and washing to an amount of the Fab region-containing peptide loaded on the affinity separation matrix, but the method is not restricted thereto. Also, a binding capacity of the affinity separation matrix to a Fab region can be expressed, for example, by a static binding capacity. Such a static binding capacity is a maximum binding capacity of the affinity separation matrix itself, and is a value not affected by flow rate or the like. In the Examples described later, the binding capacities to a Fab region were compared using 55% DBC (dynamic binding capacity) as a pseudo-static binding capacity.

The affinity separation matrix according to the present invention can effectively bind and retain IgG and an IgG fragment containing a Fab region, and thus is useful for separation and purification of such IgG and IgG fragment. The term “affinity ligand” in the disclosure means a substance and a functional group to selectively bind to and adsorb a target molecule from an aggregate of molecules on the basis of a specific affinity between molecules, such as binding between an antigen and an antibody, and means the peptide which specifically binds to a Fab region of IgG in the present invention. In the present invention, the term “ligand” also means an “affinity ligand”. In addition, the terms “affinity” and “binding capability” and the terms “affinity” and “binding affinity” have the same meaning.

It becomes possible by using the affinity separation matrix of the present invention that a peptide containing a Fab region of an immunoglobulin G is purified in accordance with affinity column chromatography purification method. A peptide containing a Fab region of an immunoglobulin can be purified by a procedure in accordance with a method for purifying an immunoglobulin by affinity column chromatography, for example, such as a method using SpA affinity separation matrix (Non-Patent Document 1). Specifically, after a buffer which contains a Fab region-containing peptide and of which pH is approximately neutral is prepared, the solution is allowed to pass through an affinity column packed with the affinity separation matrix of the present invention so that the Fab region-containing peptide is adsorbed. Then, an appropriate amount of a pure buffer is allowed to pass through the affinity column to wash the inside of the column. At the time, the target Fab region-containing peptide is still adsorbed on the affinity separation matrix of the present invention in the column. The affinity separation matrix on which the peptide according to the present invention is immobilized as a ligand is excellent in the absorption and retention performance of a target Fab region-containing peptide from the step of adding a sample through the step of washing the matrix. Then, an acid buffer of which pH is appropriately adjusted is allowed to pass through the column to elute the target Fab region-containing peptide. As a result, purification with high purity can be achieved. Into the acid buffer, a substance for promoting dissociation of a Fab region-containing peptide from the matrix may be added. The affinity separation matrix according to the present invention has high adsorption performance and binging capacity to a Fab region-containing peptide, thus can also endure washing for a long time period after allowing the Fab region-containing peptide to pass through an affinity column packed with the affinity separation matrix, and can be used also for treating a solution containing a Fab region-containing peptide in a high concentration.

The affinity separation matrix according to the present invention can be reused by allowing an adequate strong acid or strong alkaline pure buffer which does not completely impair the function of the ligand compound or the base material of the carrier to pass through the matrix for washing. In the buffer for washing, an adequate modifying agent or an organic solvent may be added.

The present application claims the benefit of the priority date of Japanese patent application No. 2014-174075 filed on Aug. 28, 2014. All of the contents of the Japanese patent application No. 2014-174075 filed on Aug. 28, 2014, are incorporated by reference herein.

### EXAMPLES

Hereinafter, the present invention is described in more detail with Examples. However, the present invention is not restricted to the following Examples.

The peptide variant obtained in the following Examples is described as “domain—introduced mutation”, and wild type into which mutation is not introduced is described as “domain—Wild”. For example, pi domain of wild SpG having SEQ ID NO: 1 or SEQ ID NO: 3 is described as “β1-Wild”, and SpG-β1 domain variant into which mutation to substitute K at the 13^{th} position by T (the mutation is described as “K13T”) is described as “β1-K13T”.

With respect to a variant having two kinds of mutations, the mutations are described together with a slash. For example, SpG-β1 domain variant into which mutations of K13T and E19I are introduced is described as “β1-K13T/E19I”.

With respect to a protein in which a plurality of single domains are connected, the number of the connected domains and “d” are added after a period. For example, a protein in which two SpG β1 domain variants having mutations of K13T and E19I are connected is described as “β1-K13T/E19I.2d”.

When a Cys residue, i.e. “C”, having a functional group for immobilization is added to a C-terminal in order to immobilize a protein on a water-insoluble carrier, one letter code of the added amino acid is added after “d”. For example, a protein in which two SpG β1 domain variants having mutations of K13T and E19I are connected and to which Cys is added at the C-terminal is described as “β1-K13T/E19I.2dC”.

### Example 1: Preparation of Fab Region-Binding Peptides

(1) Preparation of Expression Plasmids of SpG-β1 Variants

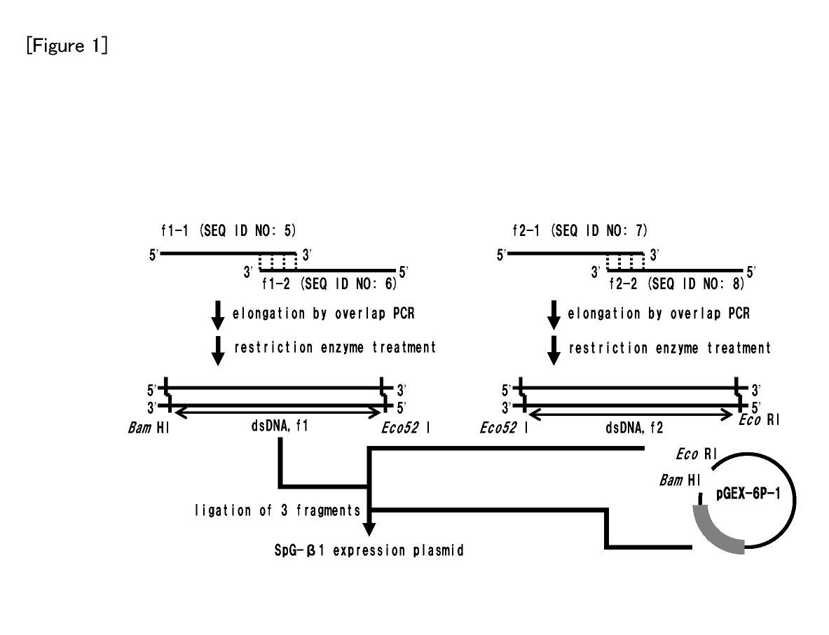

With respect to a method for preparing an expression vector, wild SpG-β1 is described as an example. A base sequence of SEQ ID NO: 4 encoding the peptide having the amino acid sequence of wild SpG-β1 (SEQ ID NO: 3) was designed by reverse translation from the amino acid sequence. The method for producing the expression plasmid is shown in FIG. 1. A DNA encoding wild SpG-β1 was prepared by ligating two kinds of double-stranded DNAs (f1 and f2) having the same restriction enzyme site, and integrated into the multiple cloning site of an expression vector. In fact, the preparation of the peptide-coding DNA and the integration into the vector were simultaneously performed by ligating three fragments for connecting three double-stranded DNAs of the two kinds of double-stranded DNAs and an expression vector. The two kinds of double-stranded DNAs were prepared by elongating two kinds of single-stranded DNAs (f1-1/f1-2 or f2-1/f2-2) respectively containing about 30-base complementary region with overlapping PCR. Hereinafter, the specific experimental procedure is described. Single-stranded oligo DNAs f1-1 (SEQ ID NO: 5)/f1-2 (SEQ ID NO: 6) were synthesized by outsourcing to Sigma Genosys. The overlapping PCR was performed using Pyrobest (manufactured by Takara Bio, Inc.) as a polymerase. The PCR product was subjected to agarose electrophoresis and the target band was cut out to extract the double-stranded DNA. The thus extracted double-stranded DNA was cleaved with the restriction enzymes BamHI and Eco52I (both available from Takara Bio, Inc.). Similarly, single-stranded oligo DNAs f2-1 (SEQ ID NO: 7)/f2-2 (SEQ ID NO: 8) were synthesized by outsourcing. The double-stranded DNA synthesized by overlapping PCR was extracted and cleaved with the restriction enzymes Eco52I and EcoRI (both available from Takara Bio, Inc.). Then, the two kinds of double-stranded DNAs were sub-cloned into the BamHI/EcoRI site in the multiple cloning site of a plasmid vector pGEX-6P-1 (GE Healthcare Bioscience). The ligation reaction for the subcloning was performed using Ligation high (manufactured by TOYOBO CO., LTD.) in accordance with the protocol attached to the product.

A competent cell (“Escherichia coli HB101” manufactured by Takara Bio, Inc.) was transformed using the above-described plasmid vector pGEX-6P-1 in accordance with the protocol attached to the competent cell product. By using the plasmid vector pGEX-6P-1, SpG-β1 which was fused with glutathione-S-transferase (hereinafter, abbreviated as “GST”) could be produced. Then, the plasmid DNA was amplified and extracted using a plasmid purification kit (“Wizard Plus SV Minipreps DNA Purification System” manufactured by Promega) in accordance with the standard protocol attached to the kit. The base sequence of the peptide-coding DNA of the expression plasmid was determined by using a DNA sequencer (“3130×1 Genetic Analyzer” manufactured by Applied Biosystems). The sequencing PCR was performed by using a gene analysis kit (“BigDye Terminator v. 1.1 Cycle Sequencing Kit” manufactured by Applied Biosystems) and DNA primers for sequencing the plasmid vector pGEX-6P-1 (manufactured by GE Healthcare Bioscience) in accordance with the attached protocol. The sequencing product was purified by using a plasmid purification kit (“BigDye XTerminator Purification Kit” manufactured by Applied Biosystems) in accordance with the attached protocol and used for the base sequence analysis.

Also, with respect to DNAs encoding various SpG-β1 variants, a base sequence encoding the peptide was designed by reverse translation from the desired amino acid sequence, and an expression plasmid and a transformant containing the peptide-coding DNA were prepared in a similar method as described above. Currently, DNA which has about 200 bases and which encodes a protein having about 60 residues can be totally synthesized by outsourcing (for example, by Eurogentec S. A.). Accordingly, SEQ ID NOs are demonstrated in the following table while corresponding to the amino acid sequences of the coded variants, and only the obtained final coding DNA sequences are demonstrated in the sequence listing.

A method for preparing two-domain type expression plasmid is also described by taking wild SpG-β1 as an example. A double-stranded DNA (f-N) was synthesize by PCR using the coding DNA part of the prepared expression plasmid of single domain SpG-β1 as a template, a primer of SEQ ID NO: 9 having BamH I recognition site added to the 5′ side and a primer of SEQ ID NO: 10 having Hind III recognition site added to the 3′ side. Similarly, a double-stranded DNA (f-C) was synthesized by PCR using a primer of SEQ ID NO: 11 having HindIII recognition site added to the 5′ side and a primer of SEQ ID NO: 12 having EcoRI recognition site added to the 3′ side. In a case of the SpG-β1 variant having a mutation at the 10^{th} position, another primer of SEQ ID NO: 13 having HindIII recognition site added to the 5′ side was used. KOD-plus (TOYOBO CO., LTD.) was used as a polymerase for PCR. The reaction product was subjected to agarose electrophoresis to extract a target double-stranded DNA. The double-stranded DNA “f-N” was cleaved with the restriction enzyme BamHI/HindIII, “f-C” was cleaved with HindIII/EcoRI, and plasmid vector pGEX-6P-1 was cleaved with the restriction enzyme BamHI/EcoRI. Expression plasmids were prepared by the same three fragment ligation procedure as described above. The subsequent transformation and confirmation of the base sequence were carried out by the same procedure as described above. Expression plasmids of various two-domain type SpG-β1 variants were prepared by a similar procedure.

(2) Preparation of Fab Region-Binding Peptides

The transformant produced by integrating each of the SpG-β1 variant gene obtained in the above-described (1) was cultured in 2×YT medium containing ampicillin at 37° C. overnight. The culture liquid was inoculated in 2×YT medium containing about 100-fold amount of ampicillin for culture at 37° C. for about 2 hours. Then, IPTG, i.e. isopropyl-1-thio-β-D-galactoside, was added so that the final concentration thereof became 0.1 mM, and the transformant was further cultured at 37° C. for 18 hours.

After the culture, the bacterial cell was collected by centrifugation and re-suspended in 5 mL of PBS buffer. The cell was broken by sonication and centrifuged to separate a supernatant fraction as a cell-free extract and an insoluble fraction. When a target gene is integrated into the multiple cloning site of pGEX-6P-1 vector, a fusion peptide having GST added to a N-terminal is produced. Each fraction was analyzed by SDS electrophoresis; as a result, a peptide band assumed to be induced by IPTG was detected at a position corresponding to a molecular weight of about 25,000 or more in the lanes of each of all the cell-free extracts obtained from all of the cultured liquids of each transformant. The molecular weights were approximately similar but the positions of bands were different depending on the kind of variant.

The GST fusion peptide was roughly purified from each of the cell-free extract containing the GST fusion peptide by affinity chromatography using a GSTrap FF column (GE Healthcare Bioscience), which had an affinity for GST. Specifically, each of the cell-free extract was added to the GSTrap FF column and the column was washed with a standard buffer (20 mM NaH₂PO₄—Na₂HPO₄, 150 mM NaCl, pH 7.4). Then, the target GST fusion peptide was eluted by using an elution buffer (50 mM Tris-HCl, 20 mM Glutathione, pH 8.0). As the sample used with fusing GST for assay in the following Examples, a peptide solution obtained by concentrating the eluent with centrifugal filter unit Amicon (manufactured by Merck Millipore) and replacing the solvent with a standard buffer was used.

When a gene is integrated into the multiple cloning site of pGEX-6P-1 vector, an amino acid sequence by which GST can be cleaved using sequence-specific protease: PreScission Protease (manufactured by GE Healthcare Bioscience) is inserted between GST and a target protein. By using such PreScission Protease, GST was cleaved in accordance with the attached protocol. The target peptide was purified by gel filtration chromatography using a Superdex 75 10/300 GL column (manufactured by GE Healthcare Bioscience) from the GST-cleaved sample used for assay. Each of the reaction mixture was added to the Superdex 75 10/300 GL column equilibrated with a standard buffer, and the target protein therein was separated and purified from the cleaved GST and PreScission Protease. Two-domain type SpG-β1 variant having a similar molecular weight to GST was purified by chromatography again from an elution fraction in a similar manner. The above-described all of the peptide purification by chromatography using the column was performed by using AKTAprime plus system (manufactured by GE Healthcare Bioscience). In addition, after the cleavage of GST, the protein produced in the present example had the sequence of Gly-Pro-Leu-Gly-Ser derived from the vector pGEX-6P-1 at the N-terminal side. A peptide in a sufficient amount for immobilization on a water-insoluble carrier was obtained by increasing the culture scale size.

### Example 2: Evaluation of Affinity of Peptide for IgG-Fab

(1) Preparation of Fab Fragment Derived from IgG (IgG-Fab)

A humanized monoclonal IgG product as a raw material was fragmented into a Fab fragment and a Fc fragment using papain, and only the Fab fragment was separated and purified. Hereinafter, a method for producing IgG-Fab derived from anti-Her2 monoclonal antibody (generic name: Trastuzumab) is described. However, other IgG-Fab such as IgG-Fab derived from anti-TNFα monoclonal antibody (generic name: Adalimumab) and anti-EGFR monoclonal antibody (generic name: Cetuximab) was basically prepared in a similar method.

Specifically, a humanized monoclonal IgG product (“HERCEPTIN” manufactured by CHUGAI PHARMACEUTICAL CO., LTD., in the case of anti-Her2 monoclonal antibody) was dissolved in a buffer for papain treatment (0.1 M AcOH—AcONa, 2 mM EDTA, 1 mM cysteine, pH 5.5), and agarose on which papain was immobilized (“Papain Agarose from papaya latex” manufactured by SIGMA) was added thereto. The mixture was incubated with stirring by a rotator at 37° C. for about 8 hours. The IgG-Fab was purified by recovering as a flow-through fraction in an affinity chromatography using KanCapA column (manufactured by KANEKA CORPORATION) from the reaction mixture which contained both of a Fab fragment and a Fc fragment and which was separated from the agarose on which papain was immobilized. The obtained IgG-Fab solution was subjected to purification by gel filtration chromatography using Superdex 75 10/300 GL column to obtain IgG-Fab solution. In the chromatography, a standard buffer was used for equilibration and separation. Similarly to the above-described Example 1(1), AKTAprime plus system was used in the chromatography for protein purification.

(2) Analysis of Affinity of SpG-β1 Variants for IgG-Fab

The affinity of each of the SpG-β1 variants fused with GST and obtained in the above Example 1(2) for IgG-Fab was evaluated using a biosensor Biacore 3000 (manufactured by GE Healthcare Bioscience) utilizing surface plasmon resonance. In the present example, the IgG-Fab obtained in the above Example 2(1) was immobilized on a sensor tip, and each of the peptide was flown on the tip to detect the interaction between the two. The IgG-Fab was immobilized on a sensor tip CM5 by amine coupling method using N-hydroxysuccinimide (NHS) and N-ethyl-N′-(3-dimethylaminopropyl)carbodiimide hydrochloride (EDC), and ethanolamine was used for blocking. All of the sensor tip and reagents for immobilization were manufactured by GE Healthcare Bioscience. The IgG-Fab solution was diluted to about 10 times using a buffer for immobilization (10 mM AcOH—AcONa, pH 4.5), and the IgG-Fab was immobilized on the sensor tip in accordance with the protocol attached to the Biacore 3000. In addition, a reference cell as negative control was also prepared by activating another flow cell on the tip with EDC/NHS and then immobilizing ethanolamine. Peptide solutions of each of the SpG-β1 variants having concentrations of 0.1 to 100 μm were prepared using a running buffer (20 mM NaH₂PO₄—Na₂HPO₄, 150 mM NaCl, 0.005% P-20, pH 7.4). The peptide solution was added to the sensor tip in a flow rate of 40 μL/min for 60 seconds. Bonding response curves at the time of addition (association phase, for 60 seconds) and after the addition (dissociation phase, for 60 seconds) were sequentially obtained at a measurement temperature of 25° C. After each measurement, 20 mM NaOH was added for 30 seconds to regenerate the sensor tip. The procedure was carried out to remove the added peptide remaining on the sensor tip, and it was confirmed that a binding activity of the immobilized human IgG was almost completely recovered. The bonding response curve obtained by subtracting the bonding response curve of the reference cell was subjected to fitting analysis by a binding model of 1:1 using a software BIA evaluation attached to the system, and affinity constant (K_{A}=kₒₙ/k_{off}) to human IgG-Fab was calculated. The result is shown in Table 1.

TABLE 1

| | | | Anti-TNF α antibody - Fab | | | | Anti-Her2 antibody - Fab | | | |
|---|---|---|---|---|---|---|---|---|---|---|
| | SEQ ID NO | | kₒₙ | k_{off} | K_{A} | | kₒₙ | k_{off} | K_{A} | |
| | Amino acid | Base | ×10⁴ | ×10⁻² | ×10⁵ | Vs. | ×10⁴ | ×10⁻² | ×10⁵ | Vs. |
| SpG variant | sequence | sequence | M⁻¹s | s⁻¹ | M⁻¹ | Wild | M⁻¹s | s⁻¹ | M⁻¹ | Wild |
| Wild type | 3 | 4 | 4.0 | 1.43 | 6.4 | — | 10.8 | 1.97 | 5.5 | — |
| K13T | 14 | 15 | 13.2 | 0.87 | 15.3 | 2.4 | 20.1 | 1.31 | 15.3 | 2.8 |
| K13T/E19I/F30L | 16 | 17 | 9.2 | 0.51 | 18.0 | 2.8 | 14.1 | 0.71 | 19.9 | 3.6 |
| K10R/K13T/K28Q/ | 18 | 19 | 12.5 | 0.58 | 21.4 | 3.4 | 19.4 | 1.12 | 17.2 | 3.2 |
| F30L/Q32R | | | | | | | | | | |
| K13T/E19I/V39I | 20 | 21 | 14.5 | 0.53 | 27.4 | 4.3 | 22.2 | 0.79 | 28.3 | 5.2 |
| K13T/F30L/D36G | 22 | 23 | 12.6 | 0.43 | 29.0 | 4.6 | 19.2 | 0.61 | 31.7 | 5.8 |

As the result shown in Table 1, it was confirmed that the association constant of the variant used as a Fab region-binding peptide in the present invention to IgG-Fab is improved, in other words, the binding affinity to IgG-Fab becomes stronger, in comparison with a wild peptide. Specifically, while the association constant of wild SpG-β1 to a Fab region was in 10⁵ M⁻¹ level, the association constant of the SpG-β1 variant according to the present invention to a Fab region was 10⁶ M⁻¹ or more. In addition, since the tendencies of improving the binding affinity to two kinds of IgG-Fab were similar, it seems that the variant of the present invention binds to a region common to various antibodies, such as a constant region, not to an antigen-binding region of IgG-Fab, of which sequence is different depending on the kind of an antibody. The above-described result is therefore regarded as a result of supporting the high versatility of the variant according to the present invention as an affinity ligand.

Since the binding affinity of GST-SpGβ1-K13T.1d to IgG-Fab is 2-fold or higher in comparison with GST-SpGβ1-Wild.1d, it can be said that the mutation of K13T independently contributes to an improvement in a binding affinity to IgG-Fab. In addition, since the mutation of F30L is observed in a plurality of variants, the mutation of F30L may largely contribute to an improvement in a binding affinity to IgG-Fab other than K13T among the mutations introduced into the above variants.

### Example 3: Analysis of Affinity of SpG-β1 Variants for IgG-Fab

The affinity of various GST-fusion type SpG-β1 variants for IgG-Fab was measured in a similar manner to the experiment of the above-described Example 2. Only one kind of IgG-Fab was used for the experiment, since the tendency of the result observed in one type of IgG-Fab was almost similarly found in another kind of IgG-Fab in the experiment of the above-described Example 2. The result is shown in Table 2.

TABLE 2

| | | | Anti-TNF α antibody - Fab | | | |
|---|---|---|---|---|---|---|
| | SEQ ID NO | | kₒₙ | k_{off} | K_{A} | |
| | Amino acid | Base | ×10⁴ | ×10⁻² | ×10⁵ | Vs. |
| SpG variant | sequence | sequence | M⁻¹s | s⁻¹ | M⁻¹ | Wild |
| Wild type | 3 | 4 | 7.63 | 2.04 | 3.7 | — |
| K13T/F30L/D36G | 22 | 23 | 13.90 | 0.60 | 23.1 | 6.2 |
| K13T/E19I/F30L/Y33F/V39I/D47H | 24 | 25 | 7.94 | 0.39 | 20.6 | 5.5 |
| K13T/V21I/A23T/F30L/E42L | 26 | 27 | 8.50 | 0.59 | 14.4 | 3.9 |
| K13T/E19I/K28R/F30L/Y33F | 28 | 29 | 8.55 | 0.42 | 20.2 | 5.4 |
| K13S/E19I/F30L | 30 | 31 | 13.70 | 0.62 | 22.3 | 6.0 |
| K13T/E19I/A23D/V39L | 32 | 33 | 8.43 | 0.62 | 13.6 | 3.6 |
| K13T/E19I/K28N/F30L/D40V | 34 | 35 | 10.80 | 0.76 | 14.3 | 3.8 |
| K13T/E19I/E27D/F30L/K31R | 36 | 37 | 15.00 | 0.55 | 27.4 | 7.3 |
| K13T/E19I/V21N/K28I/F30L | 38 | 39 | 21.40 | 0.71 | 30.1 | 8.1 |
| K13T/E19I/K28N/E42K | 40 | 41 | 13.70 | 1.24 | 11.0 | 3.0 |
| K13T/E19I/Y33F/N35Y/V39L | 42 | 43 | 9.05 | 0.49 | 18.7 | 5.0 |
| K13T/E19I/F30L/D47N | 44 | 45 | 9.26 | 0.73 | 12.7 | 3.4 |
| K13T/E19I/A23T/F30L/Y33F/D47N/K50R | 46 | 47 | 12.00 | 0.46 | 26.3 | 7.0 |
| K13T/E19I/F30L/Y33F | 48 | 49 | 10.60 | 0.67 | 15.9 | 4.3 |
| K13T/E19I/A23T/T25M/F30L | 50 | 51 | 10.20 | 0.91 | 11.2 | 3.0 |
| K13T/V21A/F30L/Y33F/D40E | 52 | 53 | 13.90 | 0.78 | 17.8 | 4.8 |
| K13T/E19I/K28N/F30L/Y33F | 54 | 55 | 8.80 | 0.61 | 14.4 | 3.9 |
| K13T/E19I/V21D/T25M/F30L/Y33F/N35F/D47A | 56 | 57 | 10.40 | 0.30 | 34.7 | 9.3 |
| K13T/E19I/K28I/F30L/N35F | 58 | 59 | 8.80 | 0.63 | 14.0 | 3.7 |
| K13T/E19I/K28I/F30L/Y33F/E42Q/D47Y | 60 | 61 | 9.10 | 0.63 | 14.5 | 3.9 |
| K13T/E15Q/E19V/F30L/V39L | 62 | 63 | 8.98 | 0.63 | 14.2 | 3.8 |
| K13T/E15T/E19I/T25M/E27G/F30L/N35Y | 64 | 65 | 8.07 | 0.63 | 12.8 | 3.4 |
| K13T/E19I/F30L/D47A/K50E | 66 | 67 | 7.09 | 0.62 | 11.4 | 3.1 |
| K13T/T18A/E19V/F30L/Y33F | 68 | 69 | 9.36 | 0.33 | 28.5 | 7.6 |
| K10R/K13T/E15Q/K28R/F30L/D36G/E42V | 70 | 71 | 11.50 | 0.35 | 33.0 | 8.8 |
| K13T/K28I/F30L/D36G/V39I | 72 | 73 | 7.90 | 0.62 | 12.8 | 3.4 |
| K13T/D22N/A23T/T25S/F30L/A48T | 74 | 75 | 10.40 | 0.98 | 10.6 | 2.8 |
| K13T/E19I/V21D/K28I/F30L/K31R/D47H | 76 | 77 | 9.50 | 0.68 | 13.9 | 3.7 |
| K13T/E15Q/E19I/E27D/V39I/D47H | 78 | 79 | 12.40 | 0.67 | 18.5 | 4.9 |
| K13T/E19I/D22E/A24T/F30L/Y33F/D47G | 80 | 81 | 6.95 | 0.53 | 13.2 | 3.5 |
| K13S/E19V/F30L/Y33F | 82 | 83 | 9.84 | 0.64 | 15.4 | 4.1 |
| K13T/E19I/F30L/Y45F/D47Y | 84 | 85 | 9.77 | 0.70 | 13.9 | 3.7 |

As the result shown in Table 2, GST-SpGβ1-K13T/F30L/D36G.1d showed about 6-fold higher binding affinity to IgG-Fab in comparison with GST-SpGβ1-Wild.1d. It can be said that the result is consistent with the fact that the binding affinity was about 5-fold in the experiment of the above-described Example 2. It can be considered to be natural that such a level of numerical deviation in binding parameters between experiments is generated by the deterioration caused by repeatedly regenerating IgG-Fab on a sensor chip or the error caused by manual operation such as adjustment of concentration.

The mutation of K13T highly contributes to the improvement in a binding affinity to IgG-Fab similarly as described above, and the mutation of K13S is also considered to have a similar effect. In addition, it can be said that the mutations of F30L, E19I and E19V highly contribute to the improvement in a binding affinity to IgG-Fab, since the mutations are also almost commonly observed in the variant having 5-fold or more binding affinity to IgG-Fab. In particular, the mutations are considered to contribute to the improvement in a binding affinity to IgG-Fab concertedly with the mutations of K13T and K13S.

### Example 4: Analysis of Affinity of SpG-β1 Variants for IgG-Fab

The affinity of SpG-β1 variants for IgG-Fab was measured in a similar manner to the experiment of the above-described Example 3. In the present experiment, the measurement was performed on the GST-cleaved type obtained by cleaving the GST. The GST-cleaved type is described as “Pep-”. In addition, not only one-domain type (Pep.1d) but also one-domain type with Cys at the C-terminal (Pep.1dC) and two-domain type with Cys at the C-terminal (Pep.2dC) were used to perform the experiment. The result is shown in Table 3.

TABLE 3

| | | | | Anti-TNF α antibody - Fab | | | |
|---|---|---|---|---|---|---|---|
| | | SEQ ID NO | | kₒₙ | k_{off} | K_{A} | |
| | | Amino acid | Base | ×10⁴ | ×10⁻² | ×10⁵ | Vs. |
| Construct | SpG variant | sequence | sequence | M⁻¹s | s | M⁻¹ | Wild |
| Pep.1d | Wild type | 3 | 4 | 3.9 | 27.00 | 1.4 | — |
| | K13T/E19I/V21D/T25M/F30L/Y33F/N35F/D47A | 56 | 57 | 10.9 | 2.57 | 42.4 | 29 |
| | K13T/T18A/E19I/V21A/K28I/F30L/Y33F/V39I | 86 | 87 | 11.1 | 2.07 | 53.4 | 37 |
| | K10R/K13T/T18A/E19I/V21D/T25M/F30L/Y33F/ | 88 | 89 | 13.8 | 1.17 | 119.0 | 83 |
| | N35F/D47A | | | | | | |
| Pep.1dC | Wild type | 3 | 4 | 2.7 | 15.3 | 1.8 | — |
| | K13T/E19I/V21D/T25M/F30L/Y33F/N35F/D47A | 56 | 57 | 7.5 | 1.37 | 54.7 | 31 |
| | K13T/T18A/E19I/V21A/K28I/F30L/Y33F/V39I | 86 | 87 | 8.8 | 0.97 | 91.1 | 51 |
| | K10R/K13T/T18A/E19I/V21D/T25M/F30L/Y33F/ | 88 | 89 | 9.8 | 0.62 | 156.0 | 88 |
| | N35F/D47A | | | | | | |
| Pep.2dC | Wild type | 3 | 4 | 1.9 | 5.68 | 3.4 | — |
| | K13T/E19I/V21D/T25M/F30L/Y33F/N35F/D47A | 56 | 57 | 5.6 | 0.58 | 96.8 | 29 |
| | K13T/T18A/E19I/V21A/K28I/F30L/Y33F/V39I | 86 | 87 | 6.4 | 0.62 | 102.0 | 30 |
| | K10R/K13T/T18A/E19I/V21D/T25M/F30L/Y33F/ | 88 | 89 | 11.7 | 0.14 | 856.0 | 253 |
| | N35F/D47A | | | | | | |

As the result shown in Table 3, the variants obtained by the present invention had a significantly higher affinity for IgG-Fab than wild type similarly to the above-described results. Since Pep-SpGβ1-Wild.1d after cleaving GST has a larger dissociation rate constant, the association constant thereof is decreased as compared to GST-SpGβ1-Wild.1d. When it is taken into consideration that the peptide is industrially produced as an affinity ligand, it is not particularly necessary to fuse GST. Accordingly, it can be said that the comparison under the condition after cleaving GST is close to the comparison under actual use condition.

Pep-SpGβ1-K13T/E19I/V21D/T25M/F30L/Y33F/N35F/D47A.1d had a significantly higher affinity for IgG-Fab than Pep-SpGβ1-Wild.1d similarly to the above Example 3. The improvement in the affinity was represented as the association constant which reached a value close to 30-fold in the case of the Pep type after cleaving GST.

In addition, other variants Pep-SpGβ1-K13T/T18A/E19I/V21A/K28I/F30L/Y33F/V39I.1d and Pep-SpGβ1-K10R/K13T/T18A/E19I/V21D/T25M/F30L/Y33F/N35F/D47A.1d obtained by the present invention also had a significantly higher affinity for IgG-Fab than Pep-SpGβ1-Wild.1d.

Further, Pep-SpGβ1-K10R/K13T/T18A/E19I/V21D/T25M/F30L/Y33F/N35F/D47A.1d had an 80-fold or higher association constant than Pep-SpGβ1-Wild.1d, and the value of K_{A} was in the order of 10⁷ M⁻¹. In the Protein G variant shown in Table 3, the mutations of E19I and Y33F are commonly observed in addition to the mutations of K13T and F30L described above. Thus, the mutations at the 19^{th} position and the 33^{rd} position, particularly E19I and Y33F, may also contribute to the improvement in a binding capability to IgG-Fab.

When one domain type constructs with Cys at the C-terminal were compared, similar results were also obtained. When two-domain type constructs with Cys at the C-terminal were compared, similar results were also obtained; however, Pep-SpGβ1-K10R/K13T/T18A/E19I/V21D/T25M/F30L/Y33F/N35F/D47A.2dC had a 200-fold or higher association constant as compared to Pep-SpGβ1-Wild.2dC.

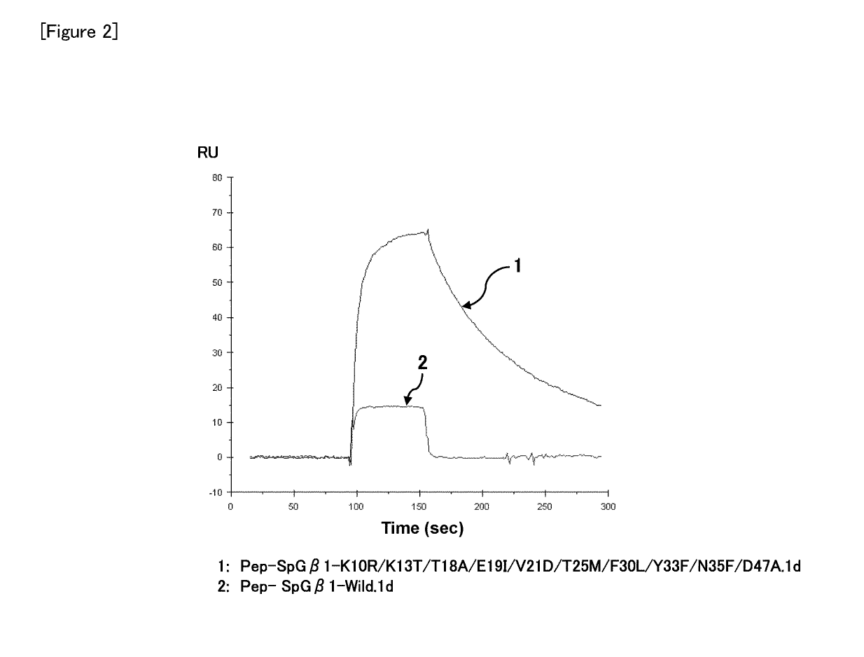

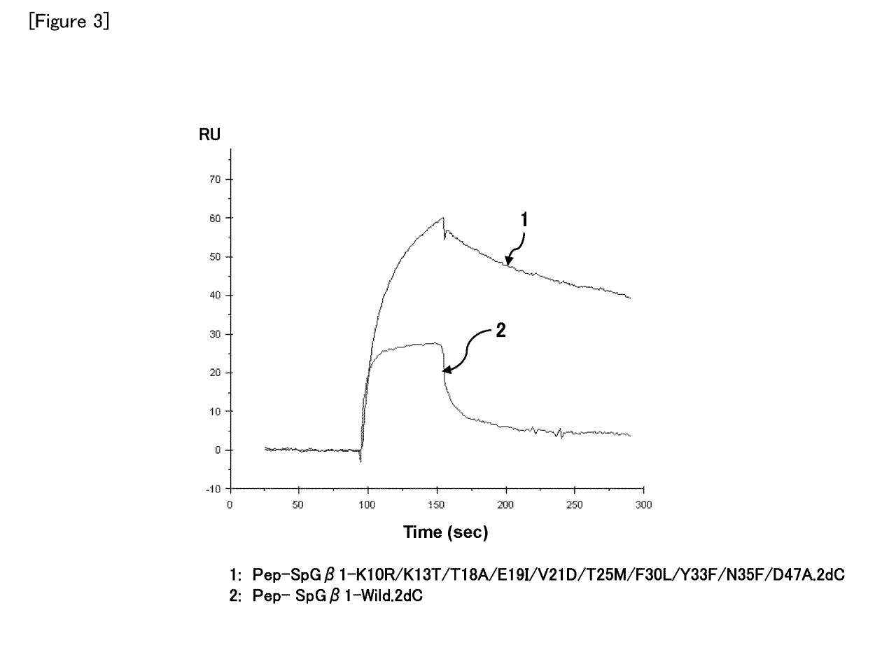

As a reference, charts in which Biacore binding reaction curves of the same type constructs of Pep.1d or Pep.2dC of wild-type SpG-β1 and K10R/K13T/T18A/E19I/V21D/T25M/F30L/Y33F/N35F/D47A in the same protein concentration of 2 μM for IgG-Fab of anti-TNFα monoclonal antibody are overlapped to be compared are shown as FIG. 2 and FIG. 3. As shown in FIG. 2 and FIG. 3, it is found that the mutated peptide according to the present invention, even in both of the cases of a domain monomeric peptide and dimeric peptide, has a higher binding capability to Fab region than wild-type SpG-β1.

### Example 5: Analysis of Affinity of Fab Region-Binding Peptide for IgG-Fab

The affinity of the peptide of SEQ ID NO: 90 for IgG-Fab was measured in a similar manner to the experiment of the above-described Example 4. In the present experiment, the measurement was performed on the GST-cleaved type after cleaving GST. The affinity was also basically evaluated similarly to the above Example 2 (2). However, the concentration of the peptide solution was adjusted to 25 nM, 100 nM or 400 nM. The analysis result is shown in Table 4.

TABLE 4

| | | Anti-EGFR antibody - Fab | | | Anti-TNF α antibody - Fab | | |
|---|---|---|---|---|---|---|---|
| SEQ ID NO | | kₒₙ | k_{off} | K_{A} | kₒₙ | k_{off} | K_{A} |
| Amino acid | Base | ×10⁴ | ×10⁻² | ×10⁶ | ×10⁴ | ×10⁻² | ×10⁶ |
| sequence | sequence | M⁻¹s | s⁻¹ | M⁻¹ | M⁻¹s | s⁻¹ | M⁻¹ |
| 90 | 91 | 25.2 | 3.9 | 65.0 | 41.3 | 1.0 | 40.0 |

As shown in Table 4, as a result of also performing the experiment for different IgG-Fab, a peptide having the amino acid sequence of SEQ ID NO: 90 obtained by introducing the mutation of E15Y into the amino acid sequence of SEQ ID NO: 86 had a high association constant for IgG-Fab as 10⁷ M⁻¹ or more. Based on the result, the amino acid mutation at the 15^{th} position, particularly E15Y, may also contribute to the improvement in a binding capability to IgG-Fab. Thus, it is considered that the peptide having the amino acid sequence of SEQ ID NO: 90 binds to Fab in a similar manner at a site different from an antigen binding site. The result may be regarded as a data showing the high versatility of the peptide.

In the following Examples, an association constant for a Fab region was not obtained. However, the following experiments were performed by selecting typical SpG-β1 variants, and all of the association constants of the SpG-β1 variants having the amino acid sequences shown in the above-described Examples 1 to 5 for a Fab region are 10⁶ M⁻¹ or more. Thus, it is assumed that the SpG-β1 variants used in the following Examples also have a similar association constant.

### Example 6: Preparation of Fab Region-Binding Peptide Immobilized Carrier

Fab region-binding peptides having constructs in which Cys was added to the C-terminal of two-domain type amino acid sequences of SEQ ID NOs: 3, 86, 88 and 90 were immobilized on a commercial water-insoluble carrier. For the immobilization, a maleimide-Cys bond was used.

First, ice-cooled 1 mM hydrochloric acid (2 mL) was flowing onto a commercial NHS activated prepacked carrier (GE Healthcare, “HiTrap NHS-Activated HP”, 1 mL) at a flow rate of about 1 mL/min. The operation was performed three times to remove an isopropanol solution in the carrier. Separately, N-[ε-maleimidocaproic acid]hydrazide.TFA (EMCH, manufactured by Thermo Fisher Scientific K.K.) was dissolved in a coupling buffer (20 mM NaH₂PO₄—Na₂HPO₄, 150 mM sodium chloride, pH 7.2) in a concentration of 10 mM. The solution (1 mL) was flown onto the carrier, and then the carrier was stood still at 25° C. for 1 hour. Next, the carrier was washed by flowing 5 mL of washing buffer A (0.5 M ethanolamine, 0.5 M sodium chloride, pH 7.2), 5 mL of the coupling buffer and 5 mL of washing buffer A in this order at a flow rate of about 1 mL/min, and then the carrier was stood still at 25° C. for 15 minutes. The carrier was further washed by flowing the coupling buffer (5 mL) at a flow rate of about 1 mL/min. Maleimide was added to the carrier in the operations described above.

Next, an operation of immobilizing the Fab region-binding peptide on the carrier to which maleimide was added was performed. Before used for immobilization, the Fab region-binding peptide was reduced under the condition of 100 mM DTT, and removal of DTT by a desalting column (GE Healthcare, “HiTrap Desalting”) and pretreatment of buffer exchange to the coupling buffer were further performed. A Fab region-binding peptide solution was flown onto the carrier to which maleimide was added at a flow rate of about 1 mL/min, and then the carrier was stood still at 25° C. for 2 hours. Then, 6 mL of the coupling buffer was flown onto the carrier in order to recover an unreacted Fab region-binding peptide. Thereafter, the carrier was washed by flowing 5 mL of washing buffer B (50 mM L-cysteine, 100 mM NaH₂PO₄—Na₂HPO₄, 0.5 M sodium chloride, pH 7.2), 5 mL of the coupling buffer and 5 mL of washing buffer B in this order at a flow rate of about 1 mL/min, and then the carrier was stood still at 25° C. for 15 minutes. The carrier was further washed by flowing the coupling buffer (5 mL) at a flow rate of about 1 mL/min. Next, the inside of the carrier was replaced with ultrapure water, and further the carrier was stored in 20% ethanol to complete preparation of a Fab region-binding peptide immobilized carrier.

The absorbency of the recovered unreacted Fab region-binding peptide at 280 nm was measured by a spectrometer, and the amount of the unreacted Fab region-binding peptide was calculated from absorption coefficient calculated from the amino acid sequence. The amount of immobilized Fab region-binding peptide was calculated from the volume of the carrier after peptide immobilization and the difference between the amount of the used Fab region-binding peptide and the calculated amount of unreacted Fab region-binding peptide. Immobilization yield was summarized in Table 5.

TABLE 5

| | Amino acid | Immobilization | Immobilization amount |
|---|---|---|---|
| Carrier No. | sequence | yield (%) | (mg/mL-gel) |
| 1 | 3 | 81.6 | 2.0 |
| 2 | 3 | 85.3 | 5.4 |
| 3 | 86 | 63.3 | 1.6 |
| 4 | 88 | 78.3 | 2.0 |
| 5 | 88 | 87.3 | 5.6 |
| 6 | 90 | 80.5 | 2.1 |
| 7 | 90 | 87.8 | 5.6 |

### Example 7: Evaluation of Binding Capacity of Fab Region-Binding Peptide Immobilized Carrier for Fab Per Ligand Density

In order to evaluate the Fab binding capacity of the Fab region-binding peptide immobilized carrier prepared in the above-described Example 6 per ligand density, 55% DBC (pseudo-static binding capacity) to Fab was measured by an affinity chromatography experiment with respect to carriers No. 1, No. 3, No. 4 and No. 6. As the Fab, a solution obtained by adjusting the concentration of the anti-TNFα antibody—Fab prepared in the above Example 2(1) to 1 mg/mL with an equilibration buffer (20 mM NaH₂PO₄—Na₂HPO₄, 150 mM sodium chloride, pH 7.4) was used. Also, 100% Abs₂₈₀ was previously measured. The 100% Abs₂₈₀ is a value of Abs₂₈₀ when 100% of the solution is passed through a cell of chromatosystem AKTAprime plus (GE Healthcare). Since the volume of the evaluated Fab region-binding peptide immobilized carrier (1 mL) was φ0.7×2.5 cm=0.96 mL, 1 mL was defined as 1 CV in a series of operations.

The Fab region-binding peptide immobilized carrier was connected to chromatosystem AKTAprime plus, and 3 CV of an equilibration buffer (20 mM NaH₂PO₄—Na₂HPO₄, 150 mM sodium chloride, pH 7.4) was flown thereon at a flow rate of 1.5 mL/min for equilibration. Next, the Fab solution was flown at a flow rate of 0.3 mL/min until the monitoring absorbency exceeded 55% of 100% Abs₂₈₀. Thereafter, 10 CV of the equilibration buffer was flown at a flow rate of 0.3 mL/min, and subsequently 3 CV of an elution buffer (50 mM citric acid, pH 2.5) was flown to elute Fab. The total amount of the Fab flown until the monitoring absorbency exceeded 55% of 100% Abs₂₈₀ was defined as Fab 55% DBC (pseudo-static binding capacity). The result obtained by dividing the value of 55% DBC of each carrier by the amount of immobilized ligand is shown in Table 6 as a binding capacity per ligand density.

TABLE 6

| | Amino acid | Immobilization amount | Biding capacity |
|---|---|---|---|
| Carrier No. | sequence | (mg/mL-gel) | per ligand density |
| 1 | 3 | 2.0 | 5.2 |
| 3 | 86 | 1.6 | 6.0 |
| 4 | 88 | 2.0 | 7.0 |
| 6 | 90 | 2.0 | 7.0 |

As shown in Table 6, a result that a binding capacity per ligand density of a carrier on which two-domain type peptides of SEQ ID NOs: 86, 88 and 90 having a high affinity for a Fab region is immobilized as a ligand is significantly improved as compared to wild-type Protein G was obtained. The result shows that the affinity separation matrix on which the Fab region-binding peptide having a high affinity for a Fab region is immobilized as a ligand has a high binding capacity to a Fab region-containing peptide.

### Example 8: Evaluation of Fab Adsorption Performance of Fab Region-Binding Peptide Immobilized Carrier

In order to evaluate a Fab adsorption performance of the Fab region-binding peptide immobilized carrier prepared in the above-described Example 6, Fab was loaded on the carrier, and the Fab recovery rate after washing was measured. As the Fab, a solution obtained by adjusting a concentration of the anti-TNFα antibody—Fab prepared in the above-described Example 2(1) to 1 mg/mL with an equilibration buffer (20 mM NaH₂PO₄—Na₂HPO₄, 150 mM sodium chloride, pH 7.4) was used. The amount of the Fab loaded on each carrier was adjusted to 50% amount of 55% DBC of each carrier.

First, in order to set the amount of the Fab loaded on each carrier, 55% DBC of each carrier was evaluated in a similar manner to the above-described Example 7. As a reference, the same evaluation was performed also for a commercial Protein G carrier (GE Healthcare, HiTrap Protein-G HP). The result is shown in Table 7.

TABLE 7

| | | Immobilization | |
|---|---|---|---|
| | Amino acid | amount | 55% DBC |
| Carrier No. | sequence | (mg/mL-gel) | (mg/mL-gel) |
| 1 | 3 | 2.0 | 10.4 |
| 2 | 3 | 5.4 | 21.0 |
| 3 | 86 | 1.6 | 9.6 |
| 4 | 88 | 2.0 | 14.0 |
| 5 | 88 | 5.6 | 29.0 |
| 6 | 90 | 2.0 | 14.0 |
| 7 | 90 | 5.6 | 27.6 |
| Commercial | — | 2.0 | 9.7 |
| HiTrap Protein-G HP | | | |

The amount of Fab to be loaded on each carrier was determined on the basis of the result shown in Table 7. For example, since 55% DBC of the carrier No. 1 was 10.4 mg/mL-gel, the amount of Fab to be loaded on the carrier No. 1 was determined as 5.2 mg.

In addition, the Fab region-binding peptide immobilized carrier was connected to chromatosystem AKTAprime plus, and 3 CV of an equilibration buffer (20 mM NaH₂PO₄—Na₂HPO₄, 150 mM sodium chloride, pH 7.4) was flown at a flow rate of 1.5 mL/min for equilibration. Next, an amount of the Fab solution respectively predetermined as described above for each carrier was flown at a flow rate of 0.3 mL/min. Then, 40 CV of the equilibration buffer was flown at a flow rate of 0.3 mL/min, and subsequently 8 CV of an elution buffer (50 mM citric acid, pH 2.5) was flown to elute Fab. A chromatochart obtained by a series of the operations was analyzed by analysis software Prime View Evaluation usable in a personal computer attached to the AKTAprime plus, and a Fab leakage area and a Fab elution area were calculated. The ratio of the Fab elution area to a total area which is the sum of the calculated Fab leakage area and Fab elution area was calculated as a recovery rate. The measurement result is shown in Table 8.

TABLE 8

| | Amino | Immobilization | Amount of | Recovery |
|---|---|---|---|---|
| | acid | amount | loaded Fab | rate |
| Carrier No. | sequence | (mg/mL-gel) | (mg) | (%) |
| 1 | 3 | 2.0 | 5.2 | 28 |
| 2 | 3 | 5.4 | 10.5 | 43 |
| 3 | 86 | 1.6 | 4.8 | 73 |
| 4 | 88 | 2.0 | 7.0 | 82 |
| 5 | 88 | 5.6 | 14.5 | 95 |
| 6 | 90 | 2.0 | 7.0 | 91 |
| 7 | 90 | 5.6 | 13.8 | 99 |
| Commercial | — | 2.0 | 4.8 | 6 |
| HiTrap | | | | |
| Protein-G HP | | | | |

The recovery rate corresponds to a rate of Fab which remained in the carrier even after washing among the loaded Fab on the carrier. When the recovery rate is high, the Fab leakage during washing is low, in other words, the Fab adsorption performance is excellent.

As shown in the result of Table 8, when the carrier No. 1 having a peptide derived from wild-type SpG-β as a ligand was compared with the Fab region-binding peptide immobilized carriers of No. 4 and No. 6 having nearly the same ligand density, it was found that in the case of the carrier which has a ligand according to the present invention having a high binding affinity to Fab region, a recovery rate is remarkably improved, in other words, an adsorption performance is improved. In addition, it was found that even in the case of the same Fab region-binding peptide, when a ligand density is higher, a recovery rate is further improved. Furthermore, the effect of improvement in the Fab recovery rate due to increase of the ligand density, in other words, the effect of improvement in Fab adsorption performance, can be also confirmed by carriers on which two-domain types of SEQ ID NO: 3, SEQ ID NO: 88 and SEQ ID NO: 90 are immobilized, and it was demonstrated that the effect is effective even when the strength of Fab affinity for the Fab region-binding peptides is different. Further, it was also demonstrated that the prepared Fab region-binding peptide immobilized carrier has higher Fab adsorption performance as compared to HiTrap Protein-G HP (GE Healthcare), which is a commercial product.

According to the above-described results, it was demonstrated that an affinity separation matrix having a high adsorption performance to Fab can be prepared by improving a binding affinity of an immobilized Fab region-binding peptide to a Fab region and increasing a ligand density in the affinity separation matrix using the Fab region-binding peptide as a ligand.

### Example 9: Preparation of Fab Region-Binding Peptide Immobilized Carrier

A Fab region-binding peptide having a two-domain type or three-domain type construct containing the amino acid sequence of SEQ ID NO: 90 with a linker sequence between domains and a C-terminal sequence of wild Protein G was immobilized on a commercial agarose carrier. For the immobilization, a bond between Cys added to the C-terminal of each construct and maleimide was used.

Specifically, first, 1.5 mL of a commercial NHS activated carrier (GE Healthcare, “NHS Activated Sepharose 4 Fast Flow”) was transferred onto a glass filter, and isopropanol as a storage solution was sucked to be removed, and then the carrier was washed with ice-cooled 1 mM hydrochloric acid (5 mL). Subsequently, the carrier was washed with 5 mL of a coupling buffer (20 mM NaH₂PO₄—Na₂HPO₄, 150 mM sodium chloride, pH 7.2), and then recovered and transferred into a centrifuge tube while suspending in the coupling buffer. In the coupling buffer, N-[ε-maleimidocaproic acid]hydrazide.TFA (EMCH, Thermo Fisher Scientific K.K.) was dissolved. The concentration thereof was adjusted to 10 mM. The solution was added into the centrifuge tube into which the carrier was transferred to react the mixture at 25° C. for 1 hour. Then, the carrier was transferred onto a glass filter, and washed with 10 mL of washing buffer A (0.5 M ethanolamine, 0.5 M sodium chloride, pH 7.2), 10 mL of the coupling buffer and 10 mL of washing buffer A in this order, and then the carrier was stood still at 25° C. for 15 minutes. The carrier was further washed with the coupling buffer (10 mL). Maleimide was added to the carrier in the operations described above.

Next, the Fab region-binding peptide was immobilized on the carrier to which maleimide was added. The Fab region-binding peptide was pretreated in a similar manner to the above Example 6 before used for immobilization. The carrier to which maleimide was added was transferred into a centrifuge tube, and a Fab region-binding peptide solution was further added thereto to react the carrier at 25° C. for 2 hours. Thereafter, the reacted carrier was transferred onto a glass filter, and washed with 7 mL of the coupling buffer to recover an unreacted Fab region-binding peptide. Then, the carrier was washed with 10 mL of washing buffer B (50 mM L-cysteine, 100 mM NaH₂PO₄—Na₂HPO₄, 0.5 M sodium chloride, pH 7.2), 10 mL of the coupling buffer and 10 mL of washing buffer B in this order, and then the carrier was stood still at 25° C. for 15 minutes. Furthermore, the carrier was washed with 10 mL of the coupling buffer, 10 mL of ultrapure water and 10 mL of 20% ethanol, and then the carrier was suspended in 20% ethanol to be recovered as a Fab region-binding peptide immobilized carrier.

The absorbency of the recovered unreacted Fab region-binding peptide at 280 nm was measured by a spectrometer, and the amount of the unreacted Fab region-binding peptide was calculated from the absorbance coefficient calculated from the amino acid sequence. The amount of immobilized Fab region-binding peptide was calculated from the volume of the carrier after peptide immobilization and the difference between the charged amount of the Fab region-binding peptide and the calculated amount of unreacted Fab region-binding peptide. The amount of the immobilized Fab region-binding peptide of each carrier was summarized in Table 9.

TABLE 9

| Carrier | | Immobilization |
|---|---|---|
| No. | Number of domain | amount (mg/mL-gel) |
| 8 | 2 | 5.9 |
| 9 | 2 | 18.5 |
| 10 | 3 | 5.4 |

### Example 10: Evaluation of Binding Capacity of Fab Region-Binding Peptide Immobilized Carrier to Fab

In order to evaluate the Fab binding capacity of the Fab region-binding peptide immobilized carrier prepared in the above-described Example 6, 55% DBC (pseudo-static binding capacity) of the carriers No. 8, No. 9 and No. 10 for Fab was measured by an affinity chromatography experiment. Tricorn 5/50 column (manufactured by GE Healthcare) was packed with 1 mL-gel of the Fab region-binding peptide immobilized carrier prepared in the above-described Example 6 for the measurement. As the Fab, a solution obtained by dissolving the anti-TNFα antibody-Fab prepared in the above Example 2(1) in an equilibration buffer (20 mM NaH₂PO₄—Na₂HPO₄, 150 mM sodium chloride, pH 7.4) in a concentration of 2 mg/mL was used. The measurement was performed in a similar manner to the above Example 7. The measurement result is shown in Table 10.

TABLE 10

| Carrier | | Immobilization amount | 55% DBC |
|---|---|---|---|
| No. | Number of domain | (mg/mL-gel) | (mg/mL-gel) |
| 8 | 2 | 5.9 | 30.9 |
| 9 | 2 | 18.5 | 73.9 |
| 10 | 3 | 5.4 | 28.7 |

As can be found by comparing the results of carriers No. 8 and No. 10 in Table 10, both of two-domain type and three-domain type showed high level 55% DBC. From the above result, it was revealed that even when the Fab region-binding peptide immobilized on a carrier has amino acid sequences for connecting domains or a construct to which an amino acid sequence other than domains is added to the C-terminal in addition to a plurality of domains, the carrier has a high binding capacity. Also, from the result of the carrier No. 9, it was demonstrated that an affinity separation matrix having a very high level of binding capacity can be prepared by increasing a ligand density.

### Example 11: Preparation of Fab Region-Binding Peptide Immobilized Carrier

While the carrier was prepared by immobilizing the Fab region-binding peptide on the agarose carrier in the above Example 6 and Example 9, a carrier was prepared by immobilizing the Fab region-binding peptide on a cellulose carrier in the present example. A Fab region-binding peptide having a construct in which there are two domains of the amino acid sequence of SEQ ID NO: 90 with a linker sequence between domains and a C-terminal sequence of wild Protein G and to which Cys was added at the C-terminal was immobilized on a cellulose carrier. As a cellulose carrier, highly crosslinked crystalline cellulose (manufactured by JNC Corporation, gel obtained by a method described in JP 2009-242770 A) was used. For the immobilization, an epoxy-Cys bond was used as a means for immobilizing a Fab region-binding peptide.

Specifically, 2 mL-gel of the above-described cellulose carrier was transferred onto a glass filter, and washed with 10 mL of ultrapure water three times. Then, the carrier was transferred into a centrifuge tube, and a predetermined amount of 1,4-bis(2,3-epoxypropoxy)butane was added. The mixture was stirred at 37° C. for 30 minutes. After 30 minutes, a 9.2 M aqueous solution of sodium hydroxide was added so as to have a final concentration of 1 M, and the mixture was stirred at 37° C. for 2 hours. The carrier was transferred onto a glass filter, and the reaction solution was removed under reduced pressure. Then, the carrier on the glass filter was washed with 30 mL of ultrapure water to obtain an epoxidized carrier.

Onto a glass filter, 1.5 mL of the epoxidized carrier was transferred to be washed with ultrapure water and 1.5 mL of an immobilization buffer (150 mM NaH₂PO₄, 1 mM EDTA, pH 8.5) three times. Then, the epoxidized carrier was transferred into a centrifuge tube, and the Fab region-binding peptide pretreated in a similar manner to the above Example 6 was further added to react the carrier at 37° C. for 30 minutes. After the reaction, sodium sulfate powder was added so as to have a final concentration of 0.9 M. After adding sodium sulfate, the mixture was reacted at 37° C. for 2 hours. After the reaction, the carrier was transferred onto a glass filter, and washed with 5 mL of the immobilization buffer three times to recover an unreacted Fab region-binding peptide. Next, the carrier was washed with 5 mL of ultrapure water three times, and then washed with 5 mL of a thioglycerol-containing inactivation buffer (200 mM NaHCO₃, 100 mM NaCl, 1 mM EDTA, pH 8.0) three times. The carrier was suspended in a thioglycerol-containing inactivation buffer to be recovered, and then transferred into a centrifuge tube to be reacted at 25° C. overnight. Thereafter, the carrier was transferred onto a glass filter, and washed with ultrapure water and 5 mL of a washing buffer (100 mM Tris-HCl, 150 mM NaCl, pH 8.0) three times. Then, the carrier was transferred into a centrifuge tube, and stirred at 25° C. for 20 minutes. The carrier was transferred onto a glass filter, and washed with 5 mL of ultrapure water three times. Furthermore, the carrier was washed with 10 mL of ultrapure water and 10 mL of 20% ethanol, and then suspended in 20% ethanol carrier to be recovered.

The absorbency of the recovered unreacted Fab region-binding peptide at 280 nm was measured by a spectrometer, and the amount of the unreacted Fab region-binding peptide was calculated from absorption coefficient calculated from the amino acid sequence. The amount of the immobilized Fab region-binding peptide on the prepared carrier is shown in Table 11.

TABLE 11

| Carrier No. | Immobilization amount (mg/mL-gel) |
|---|---|
| 11 | 12.6 |

### Example 12: Evaluation of Binding Capacity of Fab Region-Binding Peptide Immobilized Carrier for Fab

With respect to the Fab region-binding peptide immobilized carrier No. 11 prepared in the above Example 11, a binding capacity to two kinds of Fab was evaluated. As the Fab, a solution obtained by dissolving the anti-TNFα antibody—Fab prepared in the above Example 2(1) in an equilibration buffer (20 mM NaH₂PO₄—Na₂HPO₄, 150 mM sodium chloride, pH 7.4) in a concentration of 1 mg/mL and a solution obtained by dissolving polyclonal Fab prepared from a human polyclonal antibody (manufactured by NIHON PHARMACEUTICAL CO., LTD, “gamma globulin”) in a similar manner to the above Example 2(1) in an equilibration buffer (20 mM NaH₂PO₄—Na₂HPO₄, 150 mM sodium chloride, pH 7.4) in a concentration of 1 mg/mL were used. Since the human polyclonal antibody contains a non-adsorption component to Protein A carrier, IgG as an adsorption component was recovered by affinity chromatography using KANEKA KanCapA column (manufactured by KANEKA CORPORATION) before papain digestion in the above Example 2(1), and papain digestion was performed for the recovered IgG. In addition, 1 mL-gel of a commercial Protein G carrier (manufactured by GE Healthcare, “Protein-G Sepharose FF”) was used as a reference example.

Tricorn 5/50 column (manufactured by GE Healthcare) packed with 1 mL-gel of the carrier was connected to chromatosystem AKTAavant 25, and 3 CV of an equilibration buffer (20 mM NaH₂PO₄-Na₂HPO₄, 150 mM sodium chloride, pH 7.4) was flown at a flow rate of 0.25 mL/min for equilibration. Next, the Fab solution was flown at a flow rate of 0.25 mL/min until the monitoring absorbency exceeded 55% of 100% Abs₂₈₀. Then, 10 CV of the equilibration buffer was flown at a flow rate of 0.25 mL/min, and subsequently 3 CV of an elution buffer (50 mM citric acid, pH 2.5) was flown to elute Fab. The total amount of the Fab flown until the monitoring absorbency exceeded 55% of 100% Abs₂₈₀ was defined as 55% DBC for Fab. The measurement result is shown in Table 12.

TABLE 12

| | 55% DBC (mg/mL-gel) | |
|---|---|---|
| Carrier No. | Human polyclonal Fab | Anti-TNF α antibody Fab |
| 11 | 25.1 | 32.1 |
| Commercial Protein | 4.5 | 6.2 |
| G carrier | | |

The material of the carrier No. 11 is different from that of the water-insoluble carriers up to Example 11, and a method for immobilizing the Fab region-binding peptide is also different. However, as the result shown in Table 12, it was confirmed that the carrier No. 11 has a high binding capacity to human polyclonal Fab and anti-TNFα antibody—Fab, and the level of the capacity is higher in comparison with a commercial Protein G carrier. In addition, the result can be also regarded as a data demonstrating that the affinity separation matrix of the present invention on which the Fab region-binding peptide is immobilized has a high binding capacity to a wide variety of Fab and has high versatility.

### Example 13: Purification of Fab Contained in Escherichia coli Culture Supernatant

Using the carrier No. 11, it was confirmed whether the Fab in a solution containing contaminants can be purified or not. As a solution containing contaminants, a disrupted cell suspension of Escherichia coli was used. Specifically, Escherichia coli (manufactured by TAKARA BIO INC., “HB101”) was transformed using a pUC plasmid, and the transformant was cultured in 2YT medium at 37° C. overnight. The bacterial cell of the transformant was recovered, and the recovered bacterial cell was disrupted by a sonicator. A supernatant obtained by centrifugation was used as a contaminant-containing solution. Into the obtained contaminant-containing solution, the anti-TNFα antibody—Fab was added so as to have a final concentration of 1 mg/mL, and the mixture was used for the following measurement.

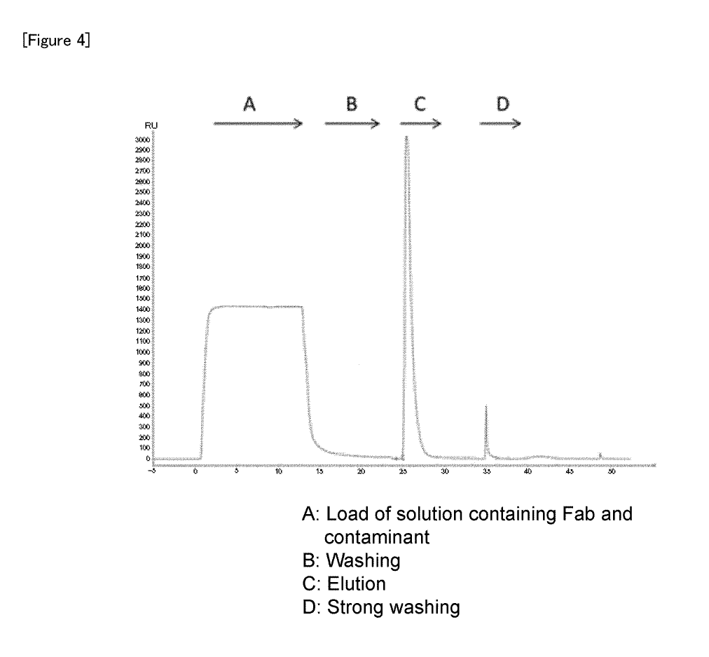

Tricorn 5/50 column packed with 1 mL-gel of the carrier No. 11 was connected to chromatosystem AKTAavant 25, and 3 CV of an equilibration buffer (20 mM NaH₂PO₄—Na₂HPO₄, 150 mM sodium chloride, pH 7.4) was flown for equilibration. Next, 12.4 mL of the above-described solution containing contaminants and Fab was flown. Then, 10 CV of the equilibration buffer was flown, and subsequently 3 CV of an elution buffer (50 mM citric acid, pH 3.0) was flown to elute Fab. Further, 3 CV of the equilibration buffer was flown, and then 10 CV of a strong washing solution (50 mM citric acid, pH 2.5) was flown. Finally, 5 CV of the equilibration buffer was flown to finish purification. A chromatochart is shown as FIG. 4. The flow rate was set to 0.25 mL/min in all processes. The solution of each process was respectively recovered, and used in SDS-PAGE analysis. The solutions of the elution process and the strong washing process were neutralized with a NaOH solution to be used.

Each of the recovered solution obtained above was subjected to SDS-PAGE using 15% polyacrylamide PRECAST GEL (manufactured by ATTO Corporation, “e-PAGEL”) in Mini-Slab Size Electrophoresis System with Integrated Power Supply PageRun (manufactured by ATTO Corporation) in accordance with the attached manual. All of the Samples were subjected to reduction treatment. A photograph of the electrophoresis gel after dyeing treatment and decoloration treatment is shown as FIG. 5.

As can be found from FIG. 5, it was confirmed that a Fab component was not present in the fraction when loaded to the carrier No. 11 and the washing fraction, and there was no Fab leakage. In addition, it was confirmed that highly pure Fab could be recovered in the elution fraction. From the result, it was demonstrated that high purity Fab can be easily obtained by one step by purification using the affinity separation matrix of the present invention on which the Fab region-binding peptide is immobilized. Furthermore, it is expected that the matrix is practically usable also in the separation or purification of antibody fragment containing a Fab region.

1. An affinity separation matrix, wherein a Fab region-binding peptide is immobilized as a ligand on a water-insoluble carrier in a density of 1.0 mg/mL-gel or more.

2. The affinity separation matrix according to claim 1, wherein the density is 5.0 mg/mL-gel or more.

3. The affinity separation matrix according to claim 1, wherein an association constant of the Fab region-binding peptide to a Fab region is 10⁶ M⁻¹ or more.

4. The affinity separation matrix according to claim 1, wherein the Fab region-binding peptide is a variant of β1 domain of Protein G.

5. The affinity separation matrix according to claim 4, wherein an amino acid sequence of the variant is an amino acid sequence derived from β1 domain of Protein G (SEQ ID NO: 3) with 3 or more substitutions of amino acid residues.

6. The affinity separation matrix according to claim 1, wherein the Fab region-binding peptide is selected from the following (1) to (3):
(1) a Fab region-binding peptide having an amino acid sequence corresponding to an amino acid sequence of SEQ ID NO: 3 derived from β1 domain of Protein G with substitution of one or more amino acid residues at positions selected from the 13^{th} position, the 15^{th} position, the 19^{th} position, the 30^{th} position and the 33^{rd} position, wherein a binding affinity to a Fab region of an immunoglobulin G is stronger than a binding affinity before introducing the substitution;
(2) a Fab region-binding peptide having the amino acid sequence specified in the (1) with deletion, substitution and/or addition of one or more amino acid residues in a region except for the 13^{th} position, the 15^{th} position, the 19^{th} position, the 30^{th} position and the 33^{rd} position, wherein a binding affinity to a Fab region of an immunoglobulin G is stronger than a binding affinity of a peptide having the amino acid sequence of SEQ ID NO: 3;
(3) a Fab region-binding peptide having an amino acid sequence with a sequence homology of 80% or more to the amino acid sequence specified in the (1), wherein a binding affinity to a Fab region of an immunoglobulin G is stronger than a binding affinity of a peptide having the amino acid sequence of SEQ ID NO: 3, provided that the amino acid residue substitution specified in the (1) at one or more positions selected from the 13^{th} position, the 15^{th} position, the 19^{th} position, the 30^{th} position and the 33^{rd} position is not further mutated in (3).

7. The affinity separation matrix according to claim 6, wherein the amino acid residue at the 13^{th} position is substituted in the amino acid sequence specified in the (1).

8. The affinity separation matrix according to claim 6, wherein the amino acid residue at the 13^{th} position is substituted by Thr or Ser in the amino acid sequence specified in the (1).

9. The affinity separation matrix according to claim 6, wherein the amino acid residue at the 30^{th} position is substituted by Val, Leu or Ile in the amino acid sequence specified in the (1).

10. The affinity separation matrix according to claim 6, wherein the amino acid residue at the 19^{th} position is substituted by Val, Leu or Ile in the amino acid sequence specified in the (1).

11. The affinity separation matrix according to claim 6, wherein the amino acid residue at the 33^{rd} position is substituted by Phe in the amino acid sequence specified in the (1).

12. The affinity separation matrix according to claim 6, wherein the amino acid residue at the 15^{th} position is substituted by Trp or Tyr in the amino acid sequence specified in the (1).

13. The affinity separation matrix according to claim 6, wherein a position of the deletion, substitution and/or addition of the amino acid residue is one or more positions selected from the 2^{nd} position, the 10^{th} position, the 18^{th} position, the 21^{st} position, the 22^{nd} position, the 23^{rd} position, the 24^{th} position, the 25^{th} position, the 27^{th} position, the 28^{th} position, the 31^{st} position, the 32^{nd} position, the 35^{th} position, the 36^{th} position, the 39^{th} position, the 40^{th} position, the 42^{nd} position, the 45^{th} position, the 47^{th} position and the 48^{th} position in the amino acid sequence specified in the (2).

14. The affinity separation matrix according to claim 6, wherein a position of the deletion, substitution and/or addition of the amino acid residue is N-terminal and/or C-terminal in the amino acid sequence specified in the (2).

15. The affinity separation matrix according to claim 6, wherein the sequence homology is 95% or more in the amino acid sequence specified in the (3).

16. The affinity separation matrix according to claim 1, wherein two or more domains formed by binding two or more of the Fab region-binding peptides are immobilized as a ligand.

17. A method for producing a protein comprising a Fab region, comprising the steps of:
contacting the affinity separation matrix according to claim 1 with a liquid sample comprising the protein comprising the Fab region; and
separating the protein comprising the Fab region bound on the affinity separation matrix from the affinity separation matrix.

18. The affinity separation matrix according to claim 6, wherein the amino acid residue at the 13^{th} position is substituted by Thr or Ser and the amino acid residue at the 30^{th} position is substituted by Val, Leu or Ile in the amino acid sequence specified in the (1).

19. The affinity separation matrix according to claim 6, wherein the amino acid residue at the 13^{th} position is substituted by Thr or Ser and the amino acid residue at the 19^{th} position is substituted by Val, Leu or Ile in the amino acid sequence specified in the (1).

20. The affinity separation matrix according to claim 6, wherein the amino acid residue at the 13^{th} position is substituted by Thr or Ser, the amino acid residue at the 19^{th} position is substituted by Val, Leu or Ile, and the amino acid residue at the 30^{th} position is substituted by Val, Leu or Ile in the amino acid sequence specified in the (1).


SEQUENCE LISTING (91 sequences)

SEQ ID NO: 1 - protein, 56 aa, Streptococcus sp. G148
DTYKLILNGKTLKGETTTEAVDAATAEKVFKQYANDNGVDGEWTYDDATKTFTVTE

SEQ ID NO: 2 - protein, 56 aa, Streptococcus sp. G148
TTYKLVINGKTLKGETTTKAVDAETAEKAFKQYANDNGVDGVWTYDDATKTFTVTE

SEQ ID NO: 3 - protein, 56 aa, Streptococcus sp. G148
TTYKLILNGKTLKGETTTEAVDAATAEKVFKQYANDNGVDGEWTYDDATKTFTVTE

SEQ ID NO: 4 - DNA, 168 bp, Artificial Sequence
  Synthetic DNA code - CDS
accacctacaaactgatcctgaacggtaagaccctgaaaggtgaaaccaccaccgaagctgttgacgctgctacggccgaaaaagtgttcaaacagtacgctaacgacaacggtgtcgacggtgaatggacctacgacgacgctaccaaaaccttcacggttaccgaa

SEQ ID NO: 5 - DNA, 60 bp, Artificial Sequence
  primer
cgtggatccaccacctacaaactgatcctgaacggtaagaccctgaaaggtgaaaccacc

SEQ ID NO: 6 - DNA, 60 bp, Artificial Sequence
  primer
ttcggccgtagcagcgtcaacagcttcggtggtggtttcacctttcagggtcttaccgtt

SEQ ID NO: 7 - DNA, 70 bp, Artificial Sequence
  primer
ctacggccgaaaaagtgttcaaacagtacgctaacgacaacggtgtcgacggtgaatggacctacgacga

SEQ ID NO: 8 - DNA, 70 bp, Artificial Sequence
  primer
cgatgaattctattcggtaaccgtgaaggttttggtagcgtcgtcgtaggtccattcaccgtcgacaccg

SEQ ID NO: 9 - DNA, 23 bp, Artificial Sequence
  primer
cctgggatccaccacctacaaac

SEQ ID NO: 10 - DNA, 32 bp, Artificial Sequence
  primer
caggataagcttgtaggtggtttcggtaaccg

SEQ ID NO: 11 - DNA, 30 bp, Artificial Sequence
  primer
acctacaagcttatcctgaacggtaagacc

SEQ ID NO: 12 - DNA, 23 bp, Artificial Sequence
  primer
cgatgaattctattcggtaaccg

SEQ ID NO: 13 - DNA, 24 bp, Artificial Sequence
  primer
acctacaagcttatcctgaacggt

SEQ ID NO: 14 - protein, 56 aa, Artificial Sequence
  Synthetic SpG mutant
  MUTAGEN (13)..(13)
TTYKLILNGKTLTGETTTEAVDAATAEKVFKQYANDNGVDGEWTYDDATKTFTVTE

SEQ ID NO: 15 - DNA, 168 bp, Artificial Sequence
  Synthetic DNA code - CDS
accacctacaaactgatcctgaacggtaagaccctgacaggtgaaaccaccaccgaagctgttgacgctgctacggccgaaaaagtgttcaaacagtacgctaacgacaacggtgtcgacggtgaatggacctacgacgacgctaccaaaaccttcacggttaccgaa

SEQ ID NO: 16 - protein, 56 aa, Artificial Sequence
  Synthetic SpG mutant
  MUTAGEN (13)..(13)
  MUTAGEN (19)..(19)
  MUTAGEN (30)..(30)
TTYKLILNGKTLTGETTTIAVDAATAEKVLKQYANDNGVDGEWTYDDATKTFTVTE

SEQ ID NO: 17 - DNA, 168 bp, Artificial Sequence
  Synthetic DNA code - CDS
accacctacaaactgatcctgaacgggaagaccctgacaggtgaaaccaccacaatagcagttgacgctgctacggccgaaaaagtgttgaaacagtacgctaacgacaacggtgtcgacggcgaatggacctacgacgacgctaccaaaaccttcacggttaccgaa

SEQ ID NO: 18 - protein, 56 aa, Artificial Sequence
  Synthetic SpG mutant
  MUTAGEN (10)..(10)
  MUTAGEN (13)..(13)
  MUTAGEN (28)..(28)
  MUTAGEN (30)..(30)
  MUTAGEN (32)..(32)
TTYKLILNGRTLTGETTTEAVDAATAEQVLKRYANDNGVDGEWTYDDATKTFTVTE

SEQ ID NO: 19 - DNA, 168 bp, Artificial Sequence
  Synthetic DNA code - CDS
accacctacaaactgatcctgaacggtaggaccctgacaggtgaaaccaccaccgaagctgttgacgctgctactgccgaacaagtgctcaaacggtacgctaacgacaacggtgtcgacggtgaatggacctacgacgacgctaccaaaaccttcacggttaccgag

SEQ ID NO: 20 - protein, 56 aa, Artificial Sequence
  Synthetic SpG mutant
  MUTAGEN (13)..(13)
  MUTAGEN (19)..(19)
  MUTAGEN (39)..(39)
TTYKLILNGKTLTGETTTIAVDAATAEKVFKQYANDNGIDGEWTYDDATKTFTVTE

SEQ ID NO: 21 - DNA, 168 bp, Artificial Sequence
  Synthetic DNA code - CDS
accacctacaaactaatcctgaacggaaagaccctgacaggtgaaaccaccaccatagctgttgacgctgctacggccgaaaaagtgttcaaacagtacgctaacgacaacggtatcgacggtgaatggacctacgacgacgctaccaaaaccttcacggttaccgag

SEQ ID NO: 22 - protein, 56 aa, Artificial Sequence
  Synthetic SpG mutant
  MUTAGEN (13)..(13)
  MUTAGEN (30)..(30)
  MUTAGEN (36)..(36)
TTYKLILNGKTLTGETTTEAVDAATAEKVLKQYANGNGVDGEWTYDDATKTFTVTE

SEQ ID NO: 23 - DNA, 168 bp, Artificial Sequence
  Synthetic DNA code - CDS
accacctacaaactgatcctgaacggtaagaccctgacaggtgaaaccaccaccgaagctgttgacgctgctacggccgaaaaagtgctcaaacagtacgctaacggcaacggtgtcgacggtgaatggacctacgacgacgctaccaaaaccttcacggttaccgag

SEQ ID NO: 24 - protein, 56 aa, Artificial Sequence
  Synthetic SpG mutant
  MUTAGEN (13)..(13)
  MUTAGEN (19)..(19)
  MUTAGEN (30)..(30)
  MUTAGEN (33)..(33)
  MUTAGEN (39)..(39)
  MUTAGEN (47)..(47)
TTYKLILNGKTLTGETTTIAVDAATAEKVLKQFANDNGIDGEWTYDHATKTFTVTE

SEQ ID NO: 25 - DNA, 168 bp, Artificial Sequence
  Synthetic DNA code - CDS
accacctacaaactgatcctgaacggcaagaccctgacaggtgaaaccaccaccatagctgttgacgctgctactgccgaaaaagtgctgaaacagttcgctaacgacaacggtatcgacggtgaatggacctacgaccacgctaccaaaaccttcacggttaccgaa

SEQ ID NO: 26 - protein, 56 aa, Artificial Sequence
  Synthetic SpG mutant
  MUTAGEN (13)..(13)
  MUTAGEN (21)..(21)
  MUTAGEN (23)..(23)
  MUTAGEN (30)..(30)
  MUTAGEN (42)..(42)
TTYKLILNGKTLTGETTTEAIDTATAEKVLKQYANDNGVDGLWTYDDATKTFTVTE

SEQ ID NO: 27 - DNA, 168 bp, Artificial Sequence
  Synthetic DNA code - CDS
accacctacaaactgatcctgaacggcaagaccctgacaggtgaaaccaccaccgaagctattgacactgctactgccgaaaaagtgctcaaacagtacgctaacgacaacggagtcgacggtctatggacctacgacgacgctaccaaaaccttcacggttaccgaa

SEQ ID NO: 28 - protein, 56 aa, Artificial Sequence
  Synthetic SpG mutant
  MUTAGEN (13)..(13)
  MUTAGEN (19)..(19)
  MUTAGEN (28)..(28)
  MUTAGEN (30)..(30)
  MUTAGEN (33)..(33)
TTYKLILNGKTLTGETTTIAVDAATAERVLKQFANDNGVDGEWTYDDATKTFTVTE

SEQ ID NO: 29 - DNA, 168 bp, Artificial Sequence
  Synthetic DNA code - CDS
accacctacaaactgatcctgaacggcaagaccctgacaggtgaaaccaccaccatagctgttgacgctgctacggccgaaagagtgctcaaacagttcgctaacgacaacggtgtcgacggtgaatggacctacgacgacgctaccaaaaccttcacggttaccgaa

SEQ ID NO: 30 - protein, 56 aa, Artificial Sequence
  Synthetic SpG mutant
  MUTAGEN (13)..(13)
  MUTAGEN (19)..(19)
  MUTAGEN (30)..(30)
TTYKLILNGKTLSGETTTIAVDAATAEKVLKQYANDNGVDGEWTYDDATKTFTVTE

SEQ ID NO: 31 - DNA, 168 bp, Artificial Sequence
  Synthetic DNA code - CDS
accacctacaaactgatcctgaacggcaagaccctgtcaggtgaaaccaccaccatagctgttgacgctgctacggccgaaaaagtgctcaaacagtacgctaacgacaacggtgtcgacggtgaatggacctatgacgacgctaccaaaaccttcacggttaccgaa

SEQ ID NO: 32 - protein, 56 aa, Artificial Sequence
  Synthetic SpG mutant
  MUTAGEN (13)..(13)
  MUTAGEN (19)..(19)
  MUTAGEN (23)..(23)
  MUTAGEN (39)..(39)
TTYKLILNGKTLTGETTTIAVDDATAEKVFKQYANDNGLDGEWTYDDATKTFTVTE

SEQ ID NO: 33 - DNA, 168 bp, Artificial Sequence
  Synthetic DNA code - CDS
accacctacaaactgatcctgaacggcaagaccctgacaggtgaaaccaccaccatagcagttgacgatgctacggccgagaaagtgttcaaacagtacgctaacgacaacggtctcgacggtgaatggacctacgacgacgctaccaaaaccttcacggttaccgaa

SEQ ID NO: 34 - protein, 56 aa, Artificial Sequence
  Synthetic SpG mutant
  MUTAGEN (13)..(13)
  MUTAGEN (19)..(19)
  MUTAGEN (28)..(28)
  MUTAGEN (30)..(30)
  MUTAGEN (40)..(40)
TTYKLILNGKTLTGETTTIAVDAATAENVLKQYANDNGVVGEWTYDDATKTFTVTE

SEQ ID NO: 35 - DNA, 168 bp, Artificial Sequence
  Synthetic DNA code - CDS
accacctacaaactgatcctgaacggcaagaccctgacaggcgaaaccaccaccatagctgttgacgctgctacggccgaaaatgtgctcaaacagtacgctaacgacaacggtgtcgtcggtgaatggacctacgacgatgctaccaaaaccttcacggttaccgaa

SEQ ID NO: 36 - protein, 56 aa, Artificial Sequence
  Synthetic SpG mutant
  MUTAGEN (13)..(13)
  MUTAGEN (19)..(19)
  MUTAGEN (27)..(27)
  MUTAGEN (30)..(30)
  MUTAGEN (31)..(31)
TTYKLILNGKTLTGETTTIAVDAATADKVLRQYANDNGVDGEWTYDDATKTFTVTE

SEQ ID NO: 37 - DNA, 168 bp, Artificial Sequence
  Synthetic DNA code - CDS
accacctacaaactgatcctgaacggcaagaccctgacaggtgaaaccaccaccatagctgttgacgctgctacggccgacaaagtgctcagacagtacgctaacgacaatggtgtcgacggtgaatggacctacgacgacgctaccaaaaccttcacggttaccgaa

SEQ ID NO: 38 - protein, 56 aa, Artificial Sequence
  Synthetic SpG mutant
  MUTAGEN (13)..(13)
  MUTAGEN (19)..(19)
  MUTAGEN (21)..(21)
  MUTAGEN (28)..(28)
  MUTAGEN (30)..(30)
TTYKLILNGKTLTGETTTIANDAATAEIVLKQYANDNGVDGEWTYDDATKTFTVTE

SEQ ID NO: 39 - DNA, 168 bp, Artificial Sequence
  Synthetic DNA code - CDS
accacctacaaactgatcctgaacggcaaaaccctgacaggtgaaaccaccaccatagctaatgacgctgctaccgccgaaatagtgctcaaacagtacgctaacgacaacggtgtcgacggagaatggacctacgacgacgctaccaaaaccttcacggttaccgaa

SEQ ID NO: 40 - protein, 56 aa, Artificial Sequence
  Synthetic SpG mutant
  MUTAGEN (13)..(13)
  MUTAGEN (19)..(19)
  MUTAGEN (28)..(28)
  MUTAGEN (42)..(42)
TTYKLILNGKTLTGETTTIAVDAATAENVFKQYANDNGVDGKWTYDDATKTFTVTE

SEQ ID NO: 41 - DNA, 168 bp, Artificial Sequence
  Synthetic DNA code - CDS
accacctacaaactgatcctgaacggcaagaccctgacaggtgaaaccacaaccatagctgtcgacgccgctacggccgaaaatgtgttcaaacagtacgctaacgacaacggcgtcgacggtaaatggacctacgacgatgctaccaaaaccttcacggttaccgaa

SEQ ID NO: 42 - protein, 56 aa, Artificial Sequence
  Synthetic SpG mutant
  MUTAGEN (13)..(13)
  MUTAGEN (19)..(19)
  MUTAGEN (33)..(33)
  MUTAGEN (35)..(35)
  MUTAGEN (39)..(39)
TTYKLILNGKTLTGETTTIAVDAATAEKVFKQFAYDNGLDGEWTYDDATKTFTVTE

SEQ ID NO: 43 - DNA, 168 bp, Artificial Sequence
  Synthetic DNA code - CDS
accacctacaaactgatcctgaacggcaagaccctgacaggtgaaaccaccaccatagctgttgacgccgctacggccgaaaaagtgttcaaacagttcgcttacgacaacggtctcgacggtgaatggacctacgacgacgctaccaaaaccttcacggttaccgaa

SEQ ID NO: 44 - protein, 56 aa, Artificial Sequence
  Synthetic SpG mutant
  MUTAGEN (13)..(13)
  MUTAGEN (19)..(19)
  MUTAGEN (30)..(30)
  MUTAGEN (47)..(47)
TTYKLILNGKTLTGETTTIAVDAATAEKVLKQYANDNGVDGEWTYDNATKTFTVTE

SEQ ID NO: 45 - DNA, 168 bp, Artificial Sequence
  Synthetic DNA code - CDS
accacctacaaactgatcctgaacggcaagaccctgacaggtgaaaccaccaccatagctgtggacgctgctacggccgagaaagtgctcaaacagtacgctaacgacaacggtgtcgacggtgaatggacctacgacaacgctaccaaaaccttcacggttaccgaa

SEQ ID NO: 46 - protein, 56 aa, Artificial Sequence
  Synthetic SpG mutant
  MUTAGEN (13)..(13)
  MUTAGEN (19)..(19)
  MUTAGEN (23)..(23)
  MUTAGEN (30)..(30)
  MUTAGEN (33)..(33)
  MUTAGEN (47)..(47)
  MUTAGEN (50)..(50)
TTYKLILNGKTLTGETTTIAVDTATAEKVLKQFANDNGVDGEWTYDNATRTFTVTE

SEQ ID NO: 47 - DNA, 168 bp, Artificial Sequence
  Synthetic DNA code - CDS
accacctacaaactgatcctgaacggcaagaccctgacaggtgaaaccaccaccatagctgttgacactgctacggccgaaaaagtgttaaaacagttcgctaacgacaacggtgtcgacggtgaatggacctacgacaacgctaccagaaccttcacggttaccgaa

SEQ ID NO: 48 - protein, 56 aa, Artificial Sequence
  Synthetic SpG mutant
  MUTAGEN (13)..(13)
  MUTAGEN (19)..(19)
  MUTAGEN (30)..(30)
  MUTAGEN (33)..(33)
TTYKLILNGKTLTGETTTIAVDAATAEKVLKQFANDNGVDGEWTYDDATKTFTVTE

SEQ ID NO: 49 - DNA, 168 bp, Artificial Sequence
  Synthetic DNA code - CDS
accacctacaaactgatcctgaacggcaagaccctgacaggtgaaaccaccaccatagctgttgacgctgctacggccgaaaaagtgctcaaacagttcgctaacgacaacggcgtcgacggtgaatggacctacgacgacgctaccaaaaccttcacggttaccgaa

SEQ ID NO: 50 - protein, 56 aa, Artificial Sequence
  Synthetic SpG mutant
  MUTAGEN (13)..(13)
  MUTAGEN (19)..(19)
  MUTAGEN (23)..(23)
  MUTAGEN (25)..(25)
  MUTAGEN (30)..(30)
TTYKLILNGKTLTGETTTIAVDTAMAEKVLKQYANDNGVDGEWTYDDATKTFTVTE

SEQ ID NO: 51 - DNA, 168 bp, Artificial Sequence
  Synthetic DNA code - CDS
accacctacaaactgatcctgaacggcaagaccctgacaggtgaaaccaccaccatagcagttgacactgctatggccgagaaagtgctcaaacagtacgctaacgacaacggcgtcgacggtgaatggacctacgacgatgctaccaaaaccttcacggttaccgaa

SEQ ID NO: 52 - protein, 56 aa, Artificial Sequence
  Synthetic SpG mutant
  MUTAGEN (13)..(13)
  MUTAGEN (21)..(21)
  MUTAGEN (30)..(30)
  MUTAGEN (33)..(33)
  MUTAGEN (40)..(40)
TTYKLILNGKTLTGETTTEAADAATAEKVLKQFANDNGVEGEWTYDDATKTFTVTE

SEQ ID NO: 53 - DNA, 168 bp, Artificial Sequence
  Synthetic DNA code - CDS
accacctacaaactgatcctgaacggcaagaccctgacaggtgaaaccaccaccgaagctgctgacgctgctacggccgaaaaagtgctcaaacagttcgctaacgacaacggtgtcgagggtgaatggacctacgacgacgctaccaaaaccttcacggttaccgaa

SEQ ID NO: 54 - protein, 56 aa, Artificial Sequence
  Synthetic SpG mutant
  MUTAGEN (13)..(13)
  MUTAGEN (19)..(19)
  MUTAGEN (28)..(28)
  MUTAGEN (30)..(30)
  MUTAGEN (33)..(33)
TTYKLILNGKTLTGETTTIAVDAATAENVLKQFANDNGVDGEWTYDDATKTFTVTE

SEQ ID NO: 55 - DNA, 168 bp, Artificial Sequence
  Synthetic DNA code - CDS
accacctacaaactgatcctgaacggcaagaccctgacaggtgaaaccaccaccatagctgttgacgctgctacggccgaaaatgtgctcaaacagttcgctaacgacaacggtgtcgacggtgaatggacctacgacgacgctaccaaaaccttcacggttaccgaa

SEQ ID NO: 56 - protein, 56 aa, Artificial Sequence
  Synthetic SpG mutant
  MUTAGEN (13)..(13)
  MUTAGEN (19)..(19)
  MUTAGEN (21)..(21)
  MUTAGEN (25)..(25)
  MUTAGEN (30)..(30)
  MUTAGEN (33)..(33)
  MUTAGEN (35)..(35)
  MUTAGEN (47)..(47)
TTYKLILNGKTLTGETTTIADDAAMAEKVLKQFAFDNGVDGEWTYDAATKTFTVTE

SEQ ID NO: 57 - DNA, 168 bp, Artificial Sequence
  Synthetic DNA code - CDS
accacctacaaactgatcctgaacggcaagaccctgacaggtgaaaccaccaccatagctgatgacgctgctatggccgaaaaagtgttgaaacagttcgctttcgacaatggtgtcgacggtgaatggacctacgacgccgctaccaaaaccttcacggttaccgaa

SEQ ID NO: 58 - protein, 56 aa, Artificial Sequence
  Synthetic SpG mutant
  MUTAGEN (13)..(13)
  MUTAGEN (19)..(19)
  MUTAGEN (28)..(28)
  MUTAGEN (30)..(30)
  MUTAGEN (35)..(35)
TTYKLILNGKTLTGETTTIAVDAATAEIVLKQYAFDNGVDGEWTYDDATKTFTVTE

SEQ ID NO: 59 - DNA, 168 bp, Artificial Sequence
  Synthetic DNA code - CDS
accacctacaaactgatcctgaacggcaagaccctgacaggtgaaaccaccaccatagctgttgacgctgctaccgccgaaatagtgctcaaacagtacgctttcgacaacggtgtcgacggtgaatggacctacgacgacgctaccaaaaccttcacggttaccgaa

SEQ ID NO: 60 - protein, 56 aa, Artificial Sequence
  Synthetic SpG mutant
  MUTAGEN (13)..(13)
  MUTAGEN (19)..(19)
  MUTAGEN (28)..(28)
  MUTAGEN (30)..(30)
  MUTAGEN (33)..(33)
  MUTAGEN (42)..(42)
  MUTAGEN (47)..(47)
TTYKLILNGKTLTGETTTIAVDAATAEIVLKQFANDNGVDGQWTYDYATKTFTVTE

SEQ ID NO: 61 - DNA, 168 bp, Artificial Sequence
  Synthetic DNA code - CDS
accacctacaaactgatcctgaacggcaagaccctgacaggtgaaaccaccaccatagctgttgacgctgctaccgccgaaatagtgctcaaacagttcgctaacgacaacggtgtcgacggtcaatggacctacgactatgctaccaaaaccttcacggttaccgaa

SEQ ID NO: 62 - protein, 56 aa, Artificial Sequence
  Synthetic SpG mutant
  MUTAGEN (13)..(13)
  MUTAGEN (15)..(15)
  MUTAGEN (19)..(19)
  MUTAGEN (30)..(30)
  MUTAGEN (39)..(39)
TTYKLILNGKTLTGQTTTVAVDAATAEKVLKQYANDNGLDGEWTYDDATKTFTVTE

SEQ ID NO: 63 - DNA, 168 bp, Artificial Sequence
  Synthetic DNA code - CDS
accacctacaaactgatcctgaacggaaagaccctgacaggtcagaccaccaccgtagctgttgacgctgctacggccgaaaaagtgctcaaacagtacgctaatgacaacggcctcgacggtgaatggacatacgacgacgctaccaaaaccttcacggttaccgaa

SEQ ID NO: 64 - protein, 56 aa, Artificial Sequence
  Synthetic SpG mutant
  MUTAGEN (13)..(13)
  MUTAGEN (15)..(15)
  MUTAGEN (19)..(19)
  MUTAGEN (25)..(25)
  MUTAGEN (27)..(27)
  MUTAGEN (30)..(30)
  MUTAGEN (35)..(35)
TTYKLILNGKTLTGTTTTIAVDAAMAGKVLKQYAYDNGVDGEWTYDDATKTFTVTE

SEQ ID NO: 65 - DNA, 168 bp, Artificial Sequence
  Synthetic DNA code - CDS
accacctacaaactaatcctgaacggcaagaccctgacaggtacaaccaccaccatagctgtagacgctgctatggctggaaaagtgctcaaacagtacgcttacgacaacggagtcgacggtgaatggacctacgacgacgctaccaaaaccttcacggttaccgaa

SEQ ID NO: 66 - protein, 56 aa, Artificial Sequence
  Synthetic SpG mutant
  MUTAGEN (13)..(13)
  MUTAGEN (19)..(19)
  MUTAGEN (30)..(30)
  MUTAGEN (47)..(47)
  MUTAGEN (50)..(50)
TTYKLILNGKTLTGETTTIAVDAATAEKVLKQYANDNGVDGEWTYDAATETFTVTE

SEQ ID NO: 67 - DNA, 168 bp, Artificial Sequence
  Synthetic DNA code - CDS
accacctacaaactgatcctgaacggcaagaccctgacaggtgaaaccaccaccatagcagttgacgctgctacggccgaaaaagtgctcaaacagtacgcaaacgacaacggtgtcgacggtgaatggacctacgatgccgctaccgaaaccttcacggttaccgaa

SEQ ID NO: 68 - protein, 56 aa, Artificial Sequence
  Synthetic SpG mutant
  MUTAGEN (13)..(13)
  MUTAGEN (18)..(18)
  MUTAGEN (19)..(19)
  MUTAGEN (30)..(30)
  MUTAGEN (33)..(33)
TTYKLILNGKTLTGETTAVAVDAATAEKVLKQFANDNGVDGEWTYDDATKTFTVTE

SEQ ID NO: 69 - DNA, 168 bp, Artificial Sequence
  Synthetic DNA code - CDS
accacctacaaactgatcctgaacggcaagaccctgacaggtgaaaccaccgccgtagctgttgacgctgctacggccgaaaaagtgctcaaacagttcgctaacgacaacggtgtcgacggtgaatggacctacgacgatgctaccaaaaccttcacggttaccgaa

SEQ ID NO: 70 - protein, 56 aa, Artificial Sequence
  Synthetic SpG mutant
  MUTAGEN (10)..(10)
  MUTAGEN (13)..(13)
  MUTAGEN (15)..(15)
  MUTAGEN (28)..(28)
  MUTAGEN (30)..(30)
  MUTAGEN (36)..(36)
  MUTAGEN (42)..(42)
TTYKLILNGRTLTGQTTTEAVDAATAERVLKQYANGNGVDGVWTYDDATKTFTVTE

SEQ ID NO: 71 - DNA, 168 bp, Artificial Sequence
  Synthetic DNA code - CDS
accacctacaaactgatcctgaacggcaggaccctgacaggtcaaaccaccaccgaagctgttgacgctgctacggccgaaagagtgctcaaacagtacgctaacggcaacggtgtcgacggtgtatggacatacgacgatgctaccaaaaccttcacggttaccgaa

SEQ ID NO: 72 - protein, 56 aa, Artificial Sequence
  Synthetic SpG mutant
  MUTAGEN (13)..(13)
  MUTAGEN (28)..(28)
  MUTAGEN (30)..(30)
  MUTAGEN (36)..(36)
  MUTAGEN (39)..(39)
TTYKLILNGKTLTGETTTEAVDAATAEIVLKQYANGNGIDGEWTYDDATKTFTVTE

SEQ ID NO: 73 - DNA, 168 bp, Artificial Sequence
  Synthetic DNA code - CDS
accacctacaaactgatcctgaacggaaagaccctgacaggtgaaaccaccaccgaagctgttgacgctgctaccgccgaaatagtgctcaaacagtacgctaacggcaacggtatcgacggtgaatggacctacgacgacgctaccaaaaccttcacggttaccgaa

SEQ ID NO: 74 - protein, 56 aa, Artificial Sequence
  Synthetic SpG mutant
  MUTAGEN (13)..(13)
  MUTAGEN (22)..(22)
  MUTAGEN (23)..(23)
  MUTAGEN (25)..(25)
  MUTAGEN (30)..(30)
  MUTAGEN (48)..(48)
TTYKLILNGKTLTGETTTEAVNTASAEKVLKQYANDNGVDGEWTYDDTTKTFTVTE

SEQ ID NO: 75 - DNA, 168 bp, Artificial Sequence
  Synthetic DNA code - CDS
accacctacaaactgatcctgaacggaaagaccctgacaggagaaaccaccaccgaagctgttaacactgcttcggccgaaaaagtgctcaaacagtacgctaacgacaacggtgtcgacggcgaatggacctacgacgacactaccaaaaccttcacggttaccgaa

SEQ ID NO: 76 - protein, 56 aa, Artificial Sequence
  Synthetic SpG mutant
  MUTAGEN (13)..(13)
  MUTAGEN (19)..(19)
  MUTAGEN (21)..(21)
  MUTAGEN (28)..(28)
  MUTAGEN (30)..(30)
  MUTAGEN (31)..(31)
  MUTAGEN (47)..(47)
TTYKLILNGKTLTGETTTIADDAATAEIVLRQYANDNGVDGEWTYDHATKTFTVTE

SEQ ID NO: 77 - DNA, 168 bp, Artificial Sequence
  Synthetic DNA code - CDS
accacctacaaactgatcctgaacggcaagaccctgacaggtgaaaccaccaccatagctgatgacgctgctactgccgagatagtgctcagacagtacgctaacgacaacggagtcgacggtgaatggacctacgaccacgctaccaaaaccttcacggttaccgaa

SEQ ID NO: 78 - protein, 56 aa, Artificial Sequence
  Synthetic SpG mutant
  MUTAGEN (13)..(13)
  MUTAGEN (15)..(15)
  MUTAGEN (19)..(19)
  MUTAGEN (27)..(27)
  MUTAGEN (39)..(39)
  MUTAGEN (47)..(47)
TTYKLILNGKTLTGQTTTIAVDAATADKVFKQYANDNGIDGEWTYDHATKTFTVTE

SEQ ID NO: 79 - DNA, 168 bp, Artificial Sequence
  Synthetic DNA code - CDS
accacctacaaactgatcctgaacgggaagaccctgacaggtcaaaccaccaccatagctgttgacgctgctacggccgataaagtgttcaaacagtacgctaacgacaacggtatcgacggcgaatggacctacgaccacgctaccaaaaccttcacggttaccgaa

SEQ ID NO: 80 - protein, 56 aa, Artificial Sequence
  Synthetic SpG mutant
  MUTAGEN (13)..(13)
  MUTAGEN (19)..(19)
  MUTAGEN (22)..(22)
  MUTAGEN (24)..(24)
  MUTAGEN (30)..(30)
  MUTAGEN (33)..(33)
  MUTAGEN (47)..(47)
TTYKLILNGKTLTGETTTIAVEATTAEKVLKQFANDNGVDGEWTYDGATKTFTVTE

SEQ ID NO: 81 - DNA, 168 bp, Artificial Sequence
  Synthetic DNA code - CDS
accacctacaaactgatcctgaacgggaagaccctgacaggtgaaaccaccaccatagctgttgaagctactacggccgaaaaagtgctcaaacagttcgctaacgacaacggtgtcgacggcgaatggacctacgacggtgctaccaaaaccttcacggttaccgaa

SEQ ID NO: 82 - protein, 56 aa, Artificial Sequence
  Synthetic SpG mutant
  MUTAGEN (13)..(13)
  MUTAGEN (19)..(19)
  MUTAGEN (30)..(30)
  MUTAGEN (33)..(33)
TTYKLILNGKTLSGETTTVAVDAATAEKVLKQFANDNGVDGEWTYDDATKTFTVTE

SEQ ID NO: 83 - DNA, 168 bp, Artificial Sequence
  Synthetic DNA code - CDS
accacctacaaactgatcctgaacggcaagaccctatcaggtgaaaccacaaccgtagctgttgacgcagctacggccgaaaaagttctcaaacagttcgctaacgacaacggtgtcgatggtgaatggacctacgacgacgctaccaaaaccttcacggttaccgaa

SEQ ID NO: 84 - protein, 56 aa, Artificial Sequence
  Synthetic SpG mutant
  MUTAGEN (13)..(13)
  MUTAGEN (19)..(19)
  MUTAGEN (30)..(30)
  MUTAGEN (45)..(45)
  MUTAGEN (47)..(47)
TTYKLILNGKTLTGETTTIAVDAATAEKVLKQYANDNGVDGEWTFDYATKTFTVTE

SEQ ID NO: 85 - DNA, 168 bp, Artificial Sequence
  Synthetic DNA code - CDS
accacctacaaactgatcctgaacggcaagaccctgacaggtgaaaccaccaccatagctgttgacgctgctacggccgaaaaagtgctcaaacaatacgctaacgacaacggtgtcgacggcgaatggaccttcgactacgctaccaaaaccttcacggttaccgaa

SEQ ID NO: 86 - protein, 56 aa, Artificial Sequence
  Synthetic SpG mutant
  MUTAGEN (13)..(13)
  MUTAGEN (18)..(18)
  MUTAGEN (19)..(19)
  MUTAGEN (21)..(21)
  MUTAGEN (28)..(28)
  MUTAGEN (30)..(30)
  MUTAGEN (33)..(33)
  MUTAGEN (39)..(39)
TTYKLILNGKTLTGETTAIAADAATAEIVLKQFANDNGIDGEWTYDDATKTFTVTE

SEQ ID NO: 87 - DNA, 168 bp, Artificial Sequence
  Synthetic DNA code - CDS
accacctacaaactgatcctgaacggcaagaccctgacaggtgaaaccaccgccatcgctgctgacgctgctacggccgaaattgtgctcaaacagttcgctaacgacaacggtatcgacggtgaatggacctacgacgacgctaccaaaaccttcacggttaccgaa

SEQ ID NO: 88 - protein, 56 aa, Artificial Sequence
  Synthetic SpG mutant
  MUTAGEN (10)..(10)
  MUTAGEN (13)..(13)
  MUTAGEN (18)..(18)
  MUTAGEN (19)..(19)
  MUTAGEN (21)..(21)
  MUTAGEN (25)..(25)
  MUTAGEN (30)..(30)
  MUTAGEN (33)..(33)
  MUTAGEN (35)..(35)
  MUTAGEN (47)..(47)
TTYKLILNGRTLTGETTAIADDAAMAEKVLKQFAFDNGVDGEWTYDAATKTFTVTE

SEQ ID NO: 89 - DNA, 168 bp, Artificial Sequence
  Synthetic DNA code - CDS
accacctacaaactgatcctgaacggcaggaccctgactggtgaaaccaccgccatagctgatgacgctgctatggccgaaaaagtgttgaaacagttcgctttcgacaatggcgtcgacggtgaatggacctacgatgccgctaccaaaaccttcacggttaccgaa

SEQ ID NO: 90 - protein, 56 aa, Artificial Sequence
  Synthetic SpG mutant
  MUTAGEN (13)..(13)
  MUTAGEN (15)..(15)
  MUTAGEN (18)..(18)
  MUTAGEN (19)..(19)
  MUTAGEN (21)..(21)
  MUTAGEN (28)..(28)
  MUTAGEN (30)..(30)
  MUTAGEN (33)..(33)
  MUTAGEN (39)..(39)
TTYKLILNGKTLTGYTTAIAADAATAEIVLKQFANDNGIDGEWTYDDATKTFTVTE

SEQ ID NO: 91 - DNA, 168 bp, Artificial Sequence
  Synthetic DNA code - CDS
accacctacaaactgatcctgaacggtaagaccctgacaggttacaccaccgccatagctgctgacgctgctacggccgaaattgtgctcaaacagttcgctaacgacaacggtatcgacggtgaatggacctacgacgacgctaccaaaaccttcacggttaccgaa